(19) United States
(12) Patent Application Publication
(10) Pub. No.: US 2019/0375835 A1
(43) Pub. Date: Dec. 12, 2019

(54) USE OF MONOCLONAL ANTIBODIES FOR THE TREATMENT OF INFLAMMATION AND BACTERIAL INFECTIONS

(71) Applicant: LABORATOIRE FRANCAIS DU FRACTIONNEMENT ET DES BIOTECHNOLOGIES, Les Ulis (FR)

(72) Inventor: Christophe De Romeuf, Lambersart (FR)

(21) Appl. No.: 16/436,537

(22) Filed: Jun. 10, 2019

Related U.S. Application Data
(62) Division of application No. 14/652,352, filed on Jun. 15, 2015, now abandoned.

(30) Foreign Application Priority Data
Dec. 17, 2012 (FR) 12/62178
Mar. 15, 2013 (FR) 13/52362

(51) Int. Cl. C07K 16/24; A61K 45/06; A61K 39/395

(52) U.S. Cl. CPC C07K 16/241; A61K 45/06; A61K 2039/505; C07K 16/24; C07K 2317/41; A61K 39/3955

(57) ABSTRACT

A composition includes monoclonal antibodies directed against a circulating proinflammatory cytokine, the antibodies having a high affinity for the FcγRIIIa receptor (CD16), in particular the fucose level of all of the antibodies of the composition being less than 60%, and preferably less than 50%, and in particular, the galactosylation level of all of the antibodies of the composition being at least 60%, for the use thereof in the context of the prevention or treatment of the early phases of inflammation. A composition including monoclonal antibodies directed against a circulating bacterial toxin, having an improved affinity for the FcγRIIIa receptor (CD16) with respect to antibodies directed against the bacterial toxin, produced in the CHO cell line, for the use thereof in the context of the prevention or treatment of the early phases of a bacterial infection linked to the release of the toxin is also described.

The present invention relates to the use of monoclonal antibodies for the treatment of inflammation, irrespective of its origin.

Inflammation, a normal defence response following a stress such as an infection, a burn, an allergy etc., is a stereotypical immune response. Inflammation results in an immediate and temporary response involving a set of cellular and molecular, local and peripheral reactions, triggered from the focus of the stress, for the purpose of limiting the stress, then resolving it. Inflammation is thus a process of prevention followed by repair, stages necessary for the stressed tissue to return to the normal function.

This inflammatory process results from the release of messenger molecules, called molecular inflammatory factors, or chemical compounds, called chemical inflammatory factors, which will in turn mobilize general responses of the organism which are systemic signals. A distinction is made between septic inflammatory responses (bacteria, viruses, parasites) and sterile responses originating from a metabolic syndrome such as diabetes, hypercholesterolelmia etc. The intensity of the inflammation depends on the infectious agent and the location of the focus, and therefore on the nature of the tissue. The reaction can be latent and then be amplified by other agents.

The normal inflammatory phenomenon, or acute inflammation, is divided into two main phases: initiation and progression.

The initiation phase is induced by the cells infected with viruses or bacteria, by the presence of foreign bodies or the accumulation of toxic molecules (radicals, lipids, cholesterol etc.). The first reactions release messages which diffuse by inducing cell death signals or even necrosis. The target cells are endothelial cells which adopt a morphological change by chemotaxis allowing infiltration of the blood plasma cells.

The phase of progression of peripheral inflammation is in fact characterized by an activation of the nerve endings which causes a vasodilation facilitating the diffusion of the molecules into the extracellular space.

The infiltrated cells are the degranulated mast cells, monocytes, macrophages, neutrophils, lymphocytes. These cells aggregate because of the high level of production of chemoattractant factors. Chemotactic molecules are captured by receptors which induce a change in the migratory properties of the cells.

The activated macrophages release a broad spectrum of mediators constituted by small glycoproteins called cytokines such as interleukin-1 (IL-1) and TNF (tumour necrosis factor) or cachectin. These mediators, the action of which is pleiotropic, orchestrate the mechanisms which contribute to the establishment of a broadened inflammation response.

The IL-1 and INFα act on the stromal cells, fibroblasts, smooth muscle cells and cause the release of a second wave of cytokines and of monocyte-attracting molecules by activating other quiescent cells.

Acute inflammation is therefore an emergency process for the organism in the event of an alteration, but this inflammation can also turn against the organism if it is activated chronically.

Chronic inflammation corresponds to a failure of acute inflammation. The persistence of inflammation will be responsible for anatomical and functional after-effects which make chronic inflammatory diseases serious.

In fact, the persistence of the secretion of proinflammatory cytokines such as TNF or certain interleukins maintaining inflammation can induce tissue and cell degradation.

The mechanism of chronicity is not yet understood. It may be to do with the persistence of the pathogenic substance. However, it is also possible that this inflammation is self-perpetuating in the absence of any pathogenic agent.

This inflammatory reaction accompanies numerous major chronic pathologies. The chronicity of the inflammation and its location in several organs is at the origin of the concept of systemic diseases, diseases in the course of which autoimmunity plays a significant role in maintaining inflammation: systemic lupus erythematosus, rheumatoid arthritis, Gougerot-Sjögren's disease, Crohn's disease, ulcerative colitis, Graves' disease (hyperthyroidism), Hashimoto's chronic thyroiditis (hyperthyroidism), Goodpasture's syndrome, pemphigus, myasthaenia, diabetes caused by insulin resistance, auto-immune haemolytic anaemia, auto-immune thrombocytopaenic purpura, scleroderma, polymyositis and dermatomyositis, Biermer's anaemia, glome rlonephritis, Wegener's disease, Horton's disease, polyarteritis nodosa and Churg and Strauss syndrome, Still's disease, atrophic polychondritis, Behret's disease, multiple sclerosis, spondylitis.

As regards bacterial infections, they can be caused by toxins, i.e. soluble toxic substances produced by bacteria, and also by fungi, protozoa or worms.

The bacterial toxins can act at very low levels and act either at membrane level, or on intracellular targets, and are among the most active biological substances. Said toxins can be divided into two main categories: exotoxins and endotoxins. Exotoxins are proteins produced by bacteria and secreted into the surrounding medium whereas endotoxins are liposaccharides, namely constituents of the outer membrane of the wall of the gram-negative bacteria released following bacterial lysis.

The prophylaxis or treatment of such infections by means of antibodies directed against said toxins is well known to a person skilled in the art.

A first preventive means is the immunization of individuals with inactived and immunogenic toxins (toxoids), in order to allow the production by the individual of memory B lymphocytes capable of being activated and intervening rapidly in the event of bacterial infection capable of releasing the toxin.

A person skilled in the art knows the means for treating said infections. There are neutralizing antibodies directed against the toxins, for example, such as those described in the documents FR 55671 and EP 0 562 132 for treating tetanus, or documents U.S. Pat. No. 7,700,738 and US20100222555 for treating botulism. For example, the current treatment of a bacterial infection with Clostridium tetani is based on the administration of an anti-tetanus serum constituted by human anti-tetanus immunoglobulins aimed at providing the patient with antibodies in the expectation that he will produce them, with tetanus toxoid for stimulating his immune system so that he produces his own antibodies, and with an antibiotic as well as muscle relaxants and sedatives.

An antibody is said to be neutralizing when it blocks the effect of the toxin, and in particular its binding to its target and/or its entry into the target cell.

Nevertheless, such antibodies do not make it possible to remove the toxin and when the latter is bound to its target cell, the antibodies become ineffective.

Thus, there is at present a real need to provide a treatment making it possible to limit the effects of chronic inflammation and to limit the effects of bacterial infection causing the release of a toxin in an organism.

Consequently, one of the purposes of the invention is to provide means for treating the organisms infected with a toxin or for preventing the harmful effects of said toxin.

Another purpose of the invention is to provide a means of removing the bacterial toxins from the contaminated organism. Another purpose of the invention is to provide a composition making it possible to eradicate the toxin and the bacterium or the microorganism which produces it.

Consequently, one of the purposes of the invention is to provide means of prevention and care of organisms subjected to inflammation.

Another purpose of the invention is to provide a means of removing the proinflammatory cytokines.

The present invention thus relates to a composition comprising monoclonal antibodies directed either against a circulating proinflammatory cytokine, or against a circulating bacterial toxin, said antibodies having a high affinity for the FcγRIIIa receptor (CD16), for the use thereof in the context of the prevention or treatment of the early phases of inflammation.

Antibodies are secreted by the cells of the immune system, the plasmocytes (also called the secreting B lymphocytes) and constitute the main immunoglobulins in the blood.

Hereafter, the terms “immunoglobulin” and “antibody” are equivalent.

An antibody binds to the antigen to which it is specific. The antigen can be soluble or membrane-bound and is constituted either by an element foreign to the organism (bacterial, viral antigen etc.) or by a constituent element of the organism (autoantigen).

According to an embodiment of the invention, the antigen is either a circulating proinflammatory cytokine, or a circulating bacterial toxin.

The structure of the immunoglobulins (Ig) is well known to a person skilled in the art.

They are tetramers constituted by two heavy chains of approximately 50 kDa each (called H chains for Heavy) and by two light chains of approximately 25 kDa each (called L chains for Light), linked to each other by intra- and intercatenary disulphide bridges.

Each chain is constituted, at the N-terminal position, by a variable region or domain, called VL in the case of the light chain, VH in the case of the heavy chain, and at the C-terminal position, by a constant region, constituted by a single domain called CL in the case of the light chain and by three or four domains named CH1, CH2, CH3, CH4, in the case of the heavy chain.

Only the IgMs and the IgEs have the domain CH4.

The constant domains are encoded by the C genes and the variable domains are encoded by the V-J genes in the case of the light chain and V-D-J in the case of the heavy chain.

The assembly of the chains which compose an antibody makes it possible to define a characteristic Y-shaped three-dimensional structure, where:
the base of the Y corresponds to the constant Fc region which is recognized by the complement and Fc receptors (RFc) in order to mediate the effector functions of the molecule, and
the ends of the arms of the Y correspond to the respective assembly of the variable region of a light chain and the variable region of a heavy chain, said ends determining the specificity of the antibody to the antigen.

More precisely, there are five heavy chain isotypes (gamma, alpha, mu, delta and epsilon) and two light chain isotypes (kappa and lambda, the lambda chains themselves being divided into two types: lambda 1 and lambda 2). It is the heavy chain which determines the immunoglobulin class. There are thus five classes of Ig: IgG for the Gamma isotype, IgA for the Alpha isotype, IgM for the Mu isotype, IgD for the Delta isotype and IgE for the Epsilon isotype.

The kappa and lambda light chains are shared by all the classes and sub-classes. In humans, the proportion of kappa and lambda produced is in a ratio of 2 to 1.

More precisely, at the level of the VH and VL domains, the sequence variability is not distributed equally. In fact, the variable regions are constituted by four very slightly variable regions named Frameworks (FR1 to FR4) between which three hypervariable regions or Complementarity Determining Regions (CDR1 to CDR3) are inserted.

Each VH and VL is composed of three CDRs and four FRs arranged from the amine terminal (Nterminal) to the carboxy terminal (Ctenninal) in the following order: FR1, CDR1, FR2, CDR2, FR3, CDR3, FR4. The variable regions of the heavy chain and of the light chain have a binding domain which interacts with the antigen. The constant regions of the antibodies can mediate the binding of the immunoglobulin, to the tissues of the host or factors, including the various cells of the immune system (for example the effector cells), and the first component (C1q) of the standard complement system. The formation of a mature functional antibody molecule can be accomplished when two proteins are expressed in stoichiometric quantity and spontaneously assemble in the correct configuration.

For more information on the structure and properties of the different immunoglobulin classes, a person skilled in the art will refer to Daniel P. Stites et al., “Basic and Clinical Immunology”, 8th edition, Appleton & Lange, Norwalk, Conn., 1994, page 71 and Chapter 6.

The term “antibody” also denotes an antigen-binding fragment. The processes for making antibodies and antigen-binding fragments are well known to a person skilled in the art (see e.g., Sambrook et al, “Molecular Cloning: A Laboratory Manual” (2nd Ed.), Cold Spring Harbor Laboratory Press (1989); Lewin, “Genes IV”, Oxford University Press, New York, (1990), and Roitt et al., “immunology” (2nd Ed.), Gower Medical Publishing, London, N.Y. (1989), WO2006/040153, WO2006/122786, and WO2003/002609).

According to the present invention, an “antigen-binding fragment” of an antibody refers to one or more portions of an antibody which retain the ability to bind specifically to an antigen.

It has been shown that the antigen-binding function of an antibody can be carried out by fragments of the total length of the antibody. Examples of binding fragments covered by the term “antigen-binding fragment” of an antibody include: (i) a Fab fragment, a monovalent fragment consisting of the VL, VH, CL and CH1 domains; (ii) a F(ab′)2 fragment, a bivalent fragment comprising two Fab fragments linked by a disulphide bridge to the hinge region; (iii) an Fd fragment consisting of the VH and CH1 domains; (iv) an Fv fragment consisting of the VL and VH domains of a single arm of an antibody, (v) a daB fragment (Ward et al., (1989) Nature 341:544-546) which consists of a VH domain; and (vi) an isolated complementarity determining region (CDR). Furthermore, although the two domains of the Fv fragment, V and VH, are encoded by separate genes, they can be joined, by recombination techniques, via a synthetic linker which makes it possible to constitute them in a single chain of proteins in which the VH and VL regions are associated in pairs in order to form monovalent molecules (known as single chain Fvs (scFv); see for example Bird et al. (1988) Science 242:423-426; and Huston et al. (1988) Proc. Natl. Acad. Sci. USA 85:5879-5883). Such single chains of antibodies are also covered by the term “antigen-binding portion” of an antibody. These antibody fragments are obtained by using conventional processes, for example the proteolytic fragmentation processes, as described in J. Goding, Monoclonal Antibodies: Principles and Practice, pp 98-118 (N.Y. Academic Press 1983), which is included here by way of reference, as well as other techniques known to a person skilled in the art. The fragments are analyzed for their usefulness in the same way as the whole antibodies.

According to a particular aspect of the invention, the antibodies are of IgG, Iga or IgD isotype.

According to yet another aspect, the antibodies are selected from the group constituted by IgG1, IgG2, IgG3, IgG4, IgM, IgA1, IgA2, IgAsec, IgD, IgE or have the constant and/or variable domains of the abovementioned immunoglobulins.

According to another aspect the antibodies are bispecific antibodies or multispecific antibodies.

According to an alternative embodiment of the invention, the antibodies of the present invention can be modified in the form of a bispecific antibody or multispecific antibody.

According to the present invention, the term “bispecific antibody” includes any agent, for example, a protein, a peptide, or a protein complex or a peptide complex, which has two different binding specificities, binding or interacting with (a) a cell surface antigen and (b) an Fc receptor on the surface of an effector cell.

The term “multispecific antibody” includes any agent, for example, a protein, a peptide, or a protein complex or a peptide complex, which has more than two different binding specificities, binding or interacting with (a) a cell surface antigen and (b) an Fc receptor on the surface of an effector cell, and (c) at least one other component. The present invention therefore includes, but is not limited to, the bispecific antibodies, the trispecific antibodies, the tetraspecific antibodies, and other multispecific antibodies which are directed against cell surface antigens, and against receptors on the effector cells. The term “bispecific antibodies” also includes the diabodies. The diabodies are bivalent bi-specific antibodies, in which the VH and VL domains are expressed on a single polypeptide chain, but using a bond which is two short to allow association between the two domains of the same chain, thus forcing the domains to be associated with the complementary domains of another chain and thus creating two antigen-binding sites (see Holliger, P., et al. (1993) Proc. Natl. Acad. Sci. USA 90:6444-6448; Poijak, R. J., et al. (1994) Structure 2:1121-1123).

The term antibody also includes different types of antibodies, for example the recombinant antibodies, murine antibodies, chimeric antibodies, humanized antibodies, human antibodies, monoclonal antibodies or a mixture of these antibodies.

According to a particular aspect, the antibodies are recombinant antibodies. The term “recombinant antibodies” as used here includes the antibodies which are prepared, expressed, created, or isolated by recombinant means, such as antibodies isolated from an animal which is transgenic with immunoglobulin genes from another species, the antibodies expressed using a recombinant expression vector transfected into a host cell, antibodies isolated from a combinatorial recombinant antibody library, or antibodies prepared, expressed, created, or isolated by any other means which involve the splicing of the immunoglobulin gene sequences into other DNA sequences.

By “murine antibodies”, is meant antibodies containing only sequences belonging to all species of mice. As an example of such an antibody, the anti-CD3 antibody (Orthoclone OKT3®, muromonab-CD3) which is the first murine monoclonal antibody allowed for therapeutic use in humans, may be mentioned.

By “chimeric antibodies”, is meant antibodies in which the sequences of the variable regions of the light chains and of the heavy chains belong to a species different from that of the sequences of the constant regions of the light chains and of the heavy chains.

For the purposes of the invention, the sequences of the variable regions of the heavy and light chains are preferentially of murine species whereas the sequences of the constant regions of the heavy and light chains belong to a non-murine species. In this regard, for the constant regions, all the families and species of non-murine mammals are capable of being used, and in particular humans, monkeys, murids (except mice), suids, bovids, equids, felids, canids or also birds, this list not being exhaustive.

Preferably, the chimeric antibodies according to the invention will contain sequences of the constant regions of the heavy and light chains of human antibodies and sequences of the variable regions of the heavy and light chains of murine antibodies. As examples of such mouse/human chimeric antibodies, rituximab (Mabthera®), an anti-CD20 and cetuximab (Erbitux®), an anti-EGFR may be mentioned.

According to the present invention, the term “humanized antibody” refers to an antibody which retains only the antigen-binding CDR regions of the parent antibodies, in combination with the human framework regions (see Waldmann, 1991, Science 252:1657).

This refers in particular to antibodies in which all or part of the sequences of the regions involved in the recognition of the antigen (the hypervariable regions (CDR: Complementarity Determining Region) and sometimes certain amino acids of the framework regions (FR)) belong to sequences of non-human origin whereas the sequences of the constant regions and of the variable regions not involved in the recognition of the antigen are of human origin. As an example of such an antibody, daclizumab (Zenapax®), the first humanized antibody to have been used clinically, may be mentioned.

Such humanized or chimeric antibodies containing the binding sites specific to the murine antibody are expected to have reduced immunogenicity when they are administered in vivo for diagnosis, for prophylactic or therapeutic applications according to the invention.

The term “human antibodies”, as used here, includes antibodies having the variable and constant regions derived from the sequences of human germline immunoglobulin. The human antibodies according to the invention can also include amino acids residues not encoded by sequences of human germinal immunoglobulins (for example, mutations introduced at random, or by site-specific in vitro mutagenesis, or by in vivo somatic mutation). Human antibodies are generated using transgenic mice bearing parts of the human immune system rather than that of mice. Fully human monoclonal antibodies can also be prepared by immunizing transgenic mice with large parts of the heavy and light chains of human immunoglobulins; cf the American patents U.S. Pat. Nos. 5,591,669, 5,598,369, 5,545,806, 5,545,807, 6,150,584, and the references cited therein, the content of the latter being incorporated by way of reference. These animals have been genetically modified in such a way that there is a functional deletion in the production of endogenous antibodies (for example murine). The animals are also modified in order to contain all or a portion of the human germinal immunoglobulin gene locus so that the immunization of these animals results in the production of fully human antibodies directed against the antigen of interest.

According to the immunization of these mice (for example XenoMouse (Abgenix), HuMAb mice (Medarex/GenPharm)), the monoclonal antibodies are prepared according to the standard hybridoma techniques. These monoclonal antibodies have human immunoglobulin amino acid sequences and thus do not cause human anti-murine antibody (HAMA) responses when they are administered to humans. The human antibodies, like any antibody according to the present invention can be monoclonal antibodies.

As an example of such an antibody, adalimumab (Humira®), an anti-TNF-α, the first human antibody to have been authorized for clinical use may be mentioned.

For the purposes of the invention, the antibodies are monoclonal. The monoclonal antibodies or the “compositions of monoclonal antibodies” refer to antibodies having a unique specificity vis-à-vis an antigen binding to a single epitope. They are the opposite of the polyclonal antibodies which are mixtures of immunoglobulins isolated from serum and which recognize a series of different epitopes on the antigen considered.

According to another particular aspect, the antibodies is a full-length antibody. According to yet another particular aspect, this full-length antibody comprises a light chain and a heavy chain.

The affinity of said antibodies can be determined by several methods, including surface plasmon resonance (SPR), using a BIAcore 2000 type device (Pharmacia Biosensor, Upsala, Sweden). The documents Malmquist M., Current Opinion in Immunology, 5:282-286 (1993); Jönsson U et al., Biotechniques, 11:620-627 (1991) and Wu et al., Proc. Natl. Acad. Sci. USA, 95:6037-6042 (1998) describe this type of measurement.

By “affinity”, is meant the sum of the binding and repulsion forces between an epitope and a paratope. This represents the ability that an antibody has to bind to an antigen. The affinity is often expressed by the affinity constant (Kd or equilibrium dissociation constant).

The affinity constant is linked to the dissociation constant (Koff or kd) and to the association constant (Kon or ka) by the relation Kd=Koff/Kon=kd/ka.

By “dissociation constant”, is meant the reaction constant associated with the dissociation of an antigen-antibody complex.

By “association constant”, is meant the reaction constant associated with the association of an antigen-antibody complex.

The different isotypes of the immunoglobulins (IgM, IgA, IgE, IgD, IgG) differ as regards their biological activity and their effector functions. There are also differences in activity between the IgA sub-classes (IgA1, IgA2) and the IgG sub-classes (IgG1, IgG2, IgG3, IgG4).

These differences depend on the receptors to which the Fc regions of the immunoglobulins bind and on the distribution of these receptors over the membrane of the effector cells.

By “receptor”, is meant the receptors with the Fc fragment (FcR). Said receptors are FcαR (IgA); FcγRI, FcγRII, FcγRIII (IgG); FcεRI, FcεRII (IgE); FcμR (IgM); FcδR (IgD); pIgR (poly-Ig receptor) and FcRn (neonatal Fc receptor).

The IgG receptors are also called CD64 (FcγRI), CD32 (FcγRII) and CD16 (FcγRIII). Eight genes coding for the FcγRs have been identified in humans, but only five code for expressed receptors (FcγRIa, FcγRIIa, FcγRIIb, FcγRIIIa, FcγRIIIb). All are receptors activating the effector cells, apart from FcγRIIb which is a receptor inhibiting the activation of the immune cells (Muta T et al., Nature, 1994, 368:70-73).

More precisely, said type I receptors are characterized by a high affinity for the immunoglobulins (Kd of 5×10⁻⁷ to 10⁻¹⁰ M) whereas types II and III are characteristic of the receptors with low affinity (Kd of less than 10⁻⁷ M).

For more information on the Fc receptors, see Ravetch and Kinet, Annual Review of Immunology, vol 9:457-492 (1991).

By “effector cell”, is meant any cell bearing an Fc receptor, such as the lymphocytes, monocytes, neutrophils, Natural Killer (NK) cells, eosinophils, basophils, mast cells, dendritic cells, Langerhans cells and platelets.

The Fc region is responsible for the effector functions of the antibody, in particular for the cytotoxic functions.

It is also this region which determines the duration of the serum half-life of the antibody.

The term “effector function” refers in particular to ADCC (Antibody-Dependent Cellular Cytotoxicity), CDC (Complement Dependent Cytotoxicity) and ADCP (Antibody-Dependent Cellular Phagocytosis). Said functions are aimed at removing the target cells from the organism.

By “target cell”, is meant any cell bearing antigens. Said antigens can be substances foreign to the organism (pathogens) or “self” molecules, in the context of auto-immune diseases for example.

ADCC is a defence mechanism of the organism mediated by the effector cells. Said cells recognize the target cells covered with specific antibodies. The RFc-Fc bond activates the effector cells, which will then destroy the target cells by apoptosis for example, following the release of perforin and granzymes by the cytoplasmic granules (Raghavan et al., Annu Rev Cell Dev Biol 12:181-220, 1996; Ravetch et al., Annu Rev Immunol 19:275-290, 2001).

Preferably, the immunoglobulin with be of the IgG type and the Fc receptor will be the FcγRIIIa receptor.

CDC refers to the lysis of the target cells in the presence of molecules of the complement.

This mechanism refers to the classical complement pathway, the alternative pathway and the lectin pathway being independent of the antibodies.

The complement system is a set of serum proteins involved in inflammation, activation of phagocytic cells and lysis of cell membranes. It is constituted by different components such as C1, C4, C5, C9, this list not being exhaustive.

A cascade of enzymatic reactions, involving some twenty proteins, is triggered following the binding of several C1qs (one of the components of C1) to different Fcs of antibodies bound to antigens present on the target cell. The different proteolytic chain reactions which result from this generate lysis by osmotic shock of the target cell (CDC).

Preferably, the immunoglobulin is of the IgG type, particularly IgG1 and IgG3 which are considered effective whereas IgG2 and IgG4 are considered only slightly active or even inactive in the activation of the classical complement pathway (Shakib F, Basic and Clinical Aspects of IgG Subclasses, Karger, 1986).

ADCP is the mechanism by which the antibodies (via their Fc fragment) will act as opsonins in order to promote the ingestion of the target cell by phagocytes (effector cells capable of phagocytosis, mainly neutrophils and macrophages). The phagocytes bound to an opsonized target cell ingest it while surrounding it with pseudopods. These fuse, and the foreign agent is then internalized (endocyted) in the phagocyte, from now on called the phagosome. Granules and lysosomes fuse with the phagosome and pour, into what has become a phagolysosome, enzymes which thus digest the target cell. The residues are then released into the extracellular medium by exocytosis (Munn D H et al., Cancer Research; 51:1117-1123, 1991).

According to one aspect, the present invention thus relates to a composition comprising monoclonal antibodies directed against a circulating proinflammatory cytokine, said antibodies having a high affinity for the FcγRIIIa receptor (CD16), for the use thereof in the context of the prevention or treatment of the early phases of inflammation.

A circulating proinflammatory cytokine according to the invention means a proinflammatory cytokine fixed in its soluble form by the antibodies of the present invention. The high affinity of the Fc region of the antibody of the invention for the FcγRIIIa receptor (CD16) allows the antibody of the invention not to be displaced by polyclonal IgG antibodies, in particular IgGs present in the blood serum.

In another aspect of the invention, the Fc region of the antibodies may not be mutated.

By high affinity, is meant an affinity at least equal to 2×10⁶ M⁻¹, as determined by Scatchard analysis or BIAcore technology (Label-free surface plasmon resonance based technology)

In this aspect of the invention the antibodies are only directed against cytokines. They are therefore not directed against other antigens such as the 5C5 antigen (tumour antigen expressed by the cells of renal carcinomas), the BCR (B Cell Receptor), an idiotype such as that of the anti-FVll inhibiting antibodies, the TCR (T Cell Receptor), CD2, CD3, CD4, CD8, CD14, CD15, CD19, CD20, CD21, CD22, CD23, CD25, CD45, CD30, CD33, CD37, CD38, CD40, CD40L, CD46, CD52, CD54, CD66 (a, b, c, d), CD74, CD80, CD86, CD126, CD138, CD154, MUC1 (Mucin 1), MUC2 (Mucin 2), MUC3 (Mucin 3), MUC4 (Mucin 4), MUC16 (Mucin 16), HM1.24 (antigen specific to the plasmocytes overexpressed in multiple myelomas), tenascin (extracellular matrix protein), GGT (gamma-glutamyl transpeptidase), VEGF (Vascular Endothelial Growth Factor) for example.

In a particular embodiment, the composition of the present invention comprises monoclonal antibodies directed against a circulating proinflammatory cytokine, having an affinity at least equal to 2×10⁶ M⁻¹, at least equal to 2×10⁷ M⁻¹, 2×10⁹ M⁻¹, or 2×10⁹ M⁻¹, as determined by Scatchard analysis or BIAcore technology.

The present invention relates in particular to a composition comprising monoclonal antibodies directed against a circulating proinflammatory cytokine, the fucose level of all of the antibodies of said composition being less than 60%, and preferably less than 50%, for the use thereof in the context of the prevention or treatment of the early phases of inflammation.

It is known to a person skilled in the art that the reduced fucosylation level of the heavy chain of the antibodies increases their affinity for the receptors of the constant parts of the antibodies (Fc receptors), in particular the Fcγ receptors of type III (FcγRIII) expressed at the surface of the NK cells and macrophages.

Whilst the literature describes the impact of the strong interaction of the antibodies with the FcγRIII as increasing the cytotoxic power of the antibodies against a target cell via the action of effector cells such as the NK cells and the macrophages, the present invention is based on the finding made by the Inventors that a cytokine recognized by an antibody with a high affinity for CD16 will be preferentially “captured” by said antibodies and is then destroyed by the macrophages, after phagocytosis of the cytokine-antibodies complex.

Such a mechanism does not enter into competition with the natural killer (NK) cells, which also express FcγRIIIs at their surface, and antibody-dependent cellular cytotoxicity (ADCC) is not involved.

The anti-cytokine antibodies according to the invention serve as bio-scrubbers allowing the removal of the cytokines.

Said antibodies of the composition according to the invention are such that:
they are directed against different epitopes of said circulating proinflammatory cytokine, or
they are all directed against the same epitope of said circulating proinflammatory cytokine,
they all have the same CDR regions, or
they all have the same amino acid sequence of the light chain and of the heavy chain.

In the invention, the antibodies contained in the composition are either of the same type, or of a different type.

By “antibodies of the same type” is meant antibodies having an identical amino acid sequence. In other words, the antibodies of the same type all have the same amino acid sequences of their heavy chains and of their light chains. However, the antibodies of the same type according to the invention can have different post-translational modifications: for example, the antibodies of the same type can have different glycosylations.

By “antibodies of a different type” is meant antibodies having different amino acid sequences. They may be either antibodies having differences in the case of all their amino acid sequences, or having differences in the case of some of their amino acid sequences, the remainder of the sequences being identical.

Thus, a composition of antibodies according to the invention, where the antibodies are of a different type comprises:
a composition of polyclonal antibodies,
a composition of antibodies having the same constant regions, but where the variable regions are different,
a composition of antibodies having the same constant regions, and the same framework (FR) regions of the variable regions, but where the hypervariable regions (CDR) are different, a composition of chimeric antibodies, having any common region, the remainder of the sequences being different.

The composition according to the invention thus comprises antibodies of the same type or of a different type, for the prevention or treatment of the early phases of inflammation.

By “early phases of inflammation”, is meant in the invention the first stages of inflammation, i.e. the initiation of inflammation, and in particular the stage of release of proinflammatory cytokines such as TNF-α, IFN-γ, IFN-α, IL-1, IL-6, IL-8, IL-12, IL-17, IL-18, GM-CSF, in a quantity greater than 1.5 times, preferentially 3 times or even greater than 5 times the normal quantity.

In yet another advantageous embodiment, the invention relates to a composition as defined previously, where each monoclonal antibody comprised in said composition has an affinity for the FcγRIII receptors at least 1.5 times greater than that of a natural antibody directed against said circulating proinflammatory cytokine.

In yet another embodiment, the invention relates to a composition as defined previously, where said proinflammatory cytokine is selected from the following proinflammatory cytokines:
TNF-α,
IL-1β,
IL-6,
IL-8,
IL-12,
IL-17
IL-18
GM-CSF

The invention relates more particularly to a composition as defined previously, where said proinflammatory cytokine is TNF-α.

Advantageously, the invention relates to a composition as defined previously, where each antibody comprised in said composition has no properties of neutralization of said circulating proinflammatory cytokine.

The antibodies of the composition according to the invention are thus capable of recognizing one or more epitopes of said circulating proinflammatory cytokine. However, although the antibodies specifically recognize the proinflammatory cytokine, the antibodies-proinflammatory cytokine interaction does not block the activity of the proinflammatory cytokine. Thus, an antibodies-proinflammatory cytokine complex, if it is not removed by the macrophage system as defined in the invention, would always be present in the organism.

The antibodies of the invention are therefore very effective, not due to their neutralizing properties, but due to their increased ability to interact with the FcγRIII receptors, and thus remove the proinflammatory cytokine from the circulation of the contaminated organism.

The invention also relates to a composition as defined previously, in combination with at least one anti-inflammatory agent.

Among the anti-inflammatory agents of the invention, the corticoids (glucocorticoids or steroidal anti-inflammatories) and the non-steroidal anti-inflammatories may be mentioned. These anti-inflammatory agents are well known to a person skilled in the art.

The present invention also relates to highly galactosylated antibodies, in particular anti-TNF-α antibodies, and the compositions containing them.

According to another aspect, the present invention also relates to the use of monoclonal antibodies directed against a circulating bacterial toxin, for the use thereof in the context of the prevention or treatment of the early phases of a bacterial infection linked to the release of said toxin.

The antibodies of the present invention fix a circulating bacterial toxin, in its soluble form.

By “bacterial infection”, is meant an infection caused by a pathogenic bacterial strain. For the purposes of the invention, said bacterial strain denotes any strain of gram-positive bacteria or any strain of gram-negative bacteria. The gram-positive/gram-negative dichotomy is based on the composition of the wall of the bacteria, that of the gram-positive bacteria being very rich in peptidoglycan, in contrast to that of the gram-negative bacteria.

The gram-positive bacteria are for example those belonging to the genera Staphylococcus, Lactobacillus, Clostridium, Enterococcus, Listeria etc.

The gram-negative bacteria are for example the bacteria of the genera Salmonella, Escherichia coli, Pseudomonas etc.

Only pathogenic bacteria lead to a bacterial infection. The pathogenicity is linked to the virulence factors of the bacterium, namely genetic factors (chromosomal or extrachromosomal), said factors being in fact responsible for the implantation of the bacterium, its multiplication or its harmful effects (linked to the release of toxins in particular).

Bacterial infection is divided into several phases. The first consists of the colonization of the host by the bacterium (for example during a deep injury by means of an object contaminated with Clostridium tetani, by injury or consumption of food contaminated with Clostridium botulinum, by the respiratory tract by Corynebacterium diphtheriae etc.), said stage being followed by the exponential growth phase of the bacteria, a phase during which said bacteria fight against the immune system and use the host's nutrients which they need in order to develop. This is followed by the release of the toxins, then the binding of the latter to their targets, resulting in an alteration in the normal functioning of the host cell (modification of the electrolyte exchanges, inhibition of protein syntheses etc.), or even the lysis of the host cell.

The bacterial infections according to the invention refer to all the infections associated with the release of any exotoxin (such as the tetanus toxin, the botulinum toxin, the diphtheria toxin etc.) or to the release of any endotoxin, into the bloodstream.

The invention relates to a composition of monoclonal antibodies directed against a circulating bacterial toxin, having an affinity for the FcγRIIIa receptor (CD16), improved with respect to antibodies directed against said bacterial toxin, produced in the CHO cell line, for the use thereof in the context of the prevention or treatment of the early phases of a bacterial infection linked to the release of said toxin.

The antibodies according to the invention act in this way during the early phases of the infection.

By “early phases of a bacterial infection”, is meant the phases corresponding to the time interval extending between the release of the toxin by the bacterium and the binding to the membrane receptor or before the penetration of the toxin into the target cell, when the target is intracellular. In other words, the early stages of these diseases correspond to the time when the toxin is free in the organism and thus when it is accessible to the antibodies.

This aspect of the invention is based on the finding made by the Inventors that it is possible, using antibodies having a high affinity for the FcγRIII receptors, to induce the rapid clearance (removal) of the circulating toxins, as soon as they are released by the bacterium.

Thus, a toxin recognized by the antibodies according to the invention will be “captured” by said antibodies and will be destroyed by the macrophages, via phagocytosis of the immune complex formed by the toxin and antibodies directed against it.

Such a mechanism does not enter into competition with the natural killer (NK) cells, which also express the FcγRIIIs at their surface, and antibody-dependent cellular cytotoxicity (ADCC) is not involved.

The anti-toxin antibodies according to the invention thus serve as “bio-scrubbers” allowing the removal of the toxins. By “bio-scrubbers”, is meant that said antibodies can remove the circulating toxins from the blood.

In an advantageous embodiment, the invention relates to a composition as defined previously, in which said monoclonal antibodies have an affinity for the FcγRIIIa receptors at least twice as great as that of an antibody produced naturally in response to the presence of the bacterial toxin in an organism and directed against said bacterial toxin, or to that of antibodies produced in the CHO cell line.

In yet another embodiment, the invention relates to a composition as defined previously, where said bacterial toxin is selected from the following toxins:
the tetanus toxin (Clostridium tetani)
the botulinum toxin (Clostridium botulinum)
the diphtheria toxin (Corynebacterium diphtheriae)
the Anthrax toxin (Bacillus anthracis)
the pertussis toxin (Bordetella pertussis),
the cholera toxin,
the Staphylococcus toxins, and
the saxitoxins.

Advantageously, the invention relates to a composition as defined previously, in which said antibodies do not necessarily have properties of neutralizing said circulating toxin. In fact, said antibodies are capable of recognizing one or more epitopes of the circulating toxin, depending on whether they are monoclonal or polyclonal and, although they specifically recognize the toxin, they do not block its activity. The antibodies of the invention are therefore very effective, not due to their neutralizing properties, but due to their increased ability to interact with the FcγRIII receptors, and thus remove the toxin from the circulation of the contaminated organism.

In another advantageous embodiment, the invention relates to an abovementioned composition, where the bacterial infection linked to the release of said toxin is selected from: tetanus, botulism, diphtheria, pertussis, anthrax, cholera, Staphylococcus infections and saxitoxin intoxications.

A person skilled in the art knows that the tetanus toxin is responsible for tetanus, the botulinum toxin is responsible for botulism, the diphtheria toxin is responsible for diphtheria, the pertussis toxin is responsible for pertussis, the Anthrax toxin is responsible for anthrax, the cholera toxin responsible for cholera can lead to pulmonary oedema producing syndromes of acute respiratory distress, the Staphylococcus toxin is responsible in particular for food poisoning, the saxitoxins are responsible for respiratory disorders and/or paralysis.

The composition of antibodies according to the invention can also be coupled with an antibiotic treatment in order to rapidly remove the toxins released during the bacterial lysis generated by the antibiotic treatment, from the circulation. The composition of antibodies according to the invention is quite particularly recommended in the context of an antibiotic treatment aimed at an infection with gram-negative bacteria which, during lysis, will release massive quantities of the endotoxins contained in their walls.

The invention also relates to a composition as defined previously, in combination with at least one antibody directed against at least one epitope of a protein expressed at the surface of the bacterium producing said circulating bacterial toxin.

Thus, the composition according to the invention comprises:
anti-toxin antibodies, as defined previously, capable of capturing a circulating toxin, and removing it by phagocytosis, and
at least one antibody capable of recognizing at least one epitope of a protein expressed at the surface of a bacterium expressing said toxin, this second antibody allowing the stimulation of the immune system and the lysis of the bacteria.

In fact, certain bacteria are pathogenic due to the release of their toxin but also due to their simple presence in the organism. Consequently, this composition is very advantageous as it not only makes it possible to remove the toxin, which is harmful to the organism, but also to prevent the multiplication or the survival of the bacterium producing said toxin.

The antibody directed against at least one epitope of a protein expressed at the surface of the bacterium producing the toxin is preferably an antibody having an increased ability to lyse the bacterium. In other words, said antibody has a significant ability to induce ADCC.

Alternatively, the invention relates to a composition as defined previously, in combination with at least one neutralizing antibody directed against said circulating bacterial toxin.

Even more advantageously, the invention relates to a previously described composition, in combination with:
at least one antibody directed against at least one epitope of a protein expressed at the surface of the bacterium producing said circulating bacterial toxin, and
at least one neutralizing antibody directed against said circulating bacterial toxin.

The abovementioned composition therefore comprises three types of antibodies:
anti-toxin antibodies, as defined previously, capable of capturing the circulating toxin, and removing it by phagocytosis,
at least one neutralizing antibody, the effect of which is to prevent the circulating toxin from exerting its harmful effect, without however allowing the removal of the circulating toxin, and
at least one antibody capable of recognizing at least one epitope of a protein expressed at the surface of a bacterium expressing said toxin, this second antibody allowing the stimulation of the immune system and the lysis of the bacteria.

This composition is very advantageous as it makes it possible to neutralize the toxin, remove it by phagocytosis and remove the bacterium producing said toxin.

In another advantageous embodiment, the invention relates to a composition as defined previously, where said antibodies and
said antibiotic, and/or
said antibody directed against at least one epitope of a protein expressed at the surface of the bacterium producing said circulating bacterial toxin, and/or
said neutralizing antibody directed against said circulating bacterial toxin are used simultaneously, sequentially or separately over time.

In yet another advantageous embodiment, the invention relates to a previously defined composition, i.e. comprising antibodies directed either against a circulating proinflammatory cytokine, or against a circulating bacterial toxin, where said composition is used in doses varying from approximately 0.05 mg/m² to 2000 mg/m², in particular from 10 mg/m² to approximately 2000 mg/m², in particular, the unit dose administered can vary from 15 mg to approximately 3 g per patient.

The unit dose administered can vary from 100 μg to approximately 1 g per patient.

Even more advantageously, the invention relates to a composition as defined previously, said composition being in injectable form, or in spray form.

The composition of the invention is administered in particular by intravenous route, when it is presented in the form of an injectable liquid. It can be administered via the respiratory tract when it is presented in spray form.

The composition of the invention can be administered in particular by intravenous route, by sub-cutaneous route, by systemic route, by local route, by means of infiltration or orally.

The treatment can be continuous or sequential, i.e. by means of an infusion delivering said composition continuously and optionally constantly, or in discontinuous form, being taken or injected once or more than once daily, optionally repeated for several days, either consecutive, or with a latency period without treatment between administrations.

The administration of said composition can be continuous, by means of an intravenous infusion, delivering said composition either at a constant flow rate or at a variable flow rate over a few hours to several days. The administration of said composition can also be carried out discontinuously or sequentially, being taken or injected once or more than once daily, optionally repeated over several days.

In another advantageous embodiment, the invention relates to a composition as defined previously in combination with a pharmaceutically acceptable vehicle.

The antibodies of the present invention, whether directed against a circulating proinflammatory cytokine or directed against a bacterial toxin can be produced by various techniques known to a person skilled in the art, in particular those described hereafter.

The chimeric antibodies according to the invention can be prepared using genetic recombination techniques. For example, a chimeric antibody can be produced by constructing a chimeric gene comprising a sequence coding for the variable region of the heavy chain of a murine monoclonal antibody, linked by means of a linker to a sequence coding for the constant region of the heavy chain of a human antibody, and by constructing a chimeric gene comprising a sequence coding for the variable region of the light chain of a murine monoclonal antibody, linked by means of a linker to a sequence coding for the constant region of the light chain of a human antibody. By transfecting said chimeric genes, by fusion of protoplastes or any other technique, into a cell line, of murine myeloma for example, the production of chimeric mouse-human antibodies by the transformed cells is obtained. It is the document Morrison et al., Proc. Natl. Acad Sci. U.S.A., 81, pp. 6851-55 (1984) which described for the first time the preparation of such antibodies. The documents Verhoeven et al., BioEssays, 8:74, 1988, Boulianne, G. L. et al., Nature, 312:643 (1984), Sun, L. K., et al., Proc. Natl. Acad. Sci. USA 84, 214-218, U.S. Pat. Nos. 4,816,567, 6,331,415, 6,808,901 and EP 125023 can also be used for reference by a person skilled in the art, as well as that of Bobrzecka, K., et al., Immunology Letters 2, pp 151-155 which describes a procedure for fractionation of the interchain disulphide bridges of the immunoglobulins followed by an ordered rearrangement of these same disulphide bridges in order to obtain antibodies formed by rabbit Fab fragments and human Fc fragments.

Another approach to the preparation of chimeric antibodies, as described in the document FR 2 641 468, can be to graft Fab′ fragments of a murine monoclonal antibody onto human polyclonal immunoglobulins, in particular IgG, or onto Fc fragments, using a coupling agent, for example a diimide. Chimeric antibodies of the Ig-Fab′ type (also denoted Fab′-Ig), Fc-Fab′ or (Fab′)² can thus be obtained. Such chimeric antibodies are characterized by the grafting of all of the Fab′ fragment, and not only of the variable parts.

Alternatively, other authors have described obtaining monovalent chimeric antibodies by grafting Fab′ fragments of polyclonal antibodies onto IgGs or onto Fc fragments (G. T. Stevenson et al., Med. Oncol. & Tumor, 1985, Pharmacother, vol. 1, No. 4, 275-278, 1984).

The in vivo homologous recombination of the portions of the genes coding for the constant regions of the light chains and of the heavy chains of a murine immunoglobulin with portions of the genes coding for the constant regions of the light chains and of the heavy chains of a human immunoglobulin is also a means which can be used in order to obtain such antibodies (U.S. Pat. No. 5,204,244 or 5,202,238).

This list is not exhaustive.

The humanized antibodies according to the invention can also be prepared by well known techniques, such as that described for the first time in the document Jones et al., Nature. 1986, 321-522-525. This deals with the replacement of the hypervariable regions (CDRs) of a human antibody by hypervariable regions of murine origin, both in the light chains and also in the heavy chains. This technique, at present well known to a person skilled in the art by the name of “CDR grafting” has been described in numerous documents such as Singer et al., J. Immun. 150:2844-2857 (1993), Riechman et al., Nature 323:326 (1988), Verhoeyen et al., Science 239:1534 (1988) or also the U.S. Pat. Nos. 5,225,539; 5,585,089; EP 0682040 which can also be used for reference.

Most of the humanized antibodies produced by grafting of the CDR regions nevertheless have a reduced affinity with respect to a murine antibody, because of the major role of certain amino acids of the framework regions, the regions adjacent to the CDR regions.

This is why at present a person skilled in the art very often replaces not only the CDRs, but also the residues of the framework regions capable of contributing to the binding site of the antigen (Studicka et al., 1994).

Another technique which makes it possible to humanize antibodies is the technique of grafting the specificity determining regions (SDRs), which consists of no longer grafting all of the CDR regions, but only the SDR regions of the non-human antibodies into the human variable regions (Tamura et al., J Immunol. 2000; 164: 1432-41). The SDR regions are defined as the regions of the CDRs in direct contact with the antigen (Padlan et al. (1995), FASEB J. 9: 133-139). This technique therefore requires the identification of the SDRs. This can be done, for example, by determination of the 3D structure of the antigen-antibody complex, using the database of the already identified SDRs (http://paradox.harvard.edu/sdr), or by means of comparisons of the human variable sequences with those of the non-human species, using computer software such as CLUSTALW2, CLUSTALX, BLAST or FASTA.

Another alternative for obtaining humanized antibodies consists of grafting the regions known as “abbreviated CDRs”. This involves grafting the SDR regions and a few adjacent residues, upstream and downstream of the sequence. The documents De Pascalis et al., The Journal of Immunology, 2002, 169: 3076-3084; Kashmiri Syed V. S et al., Humanized Antibodies and their Applications, Volume 36, Issue, May 2005, Pages 25-34 can be used for reference.

The “variable domain resurfacing” technique, also called “veneering” as developed by ImmunoGen (U.S. Pat. No. 5,639,641) can also be used. This technology consists of giving a human “profile” to a mouse variable domain by replacing the residues exposed at the surface in the framework regions of the murine antibodies with the residues usually found at the surface of human antibodies. The documents Roguska et al., Proc Natl Acad Sci USA 1994; Mark G. E. et al. (1994) in Handbook of Experimental Pharmacology vol. 113: The pharmacology of monoclonal Antibodies, Springer-Verlag, pp 105-134 may also serve as reference.

The Germliner™ platform developed by AvantGen can also be used (http://www.avantgen.com/AvantGensTechnologiesandServices.pdf). This makes it possible to obtain humanized antibodies in which only CDR3s are of non-human origin.

This list is not exhaustive.

Obtaining the antibodies according to the invention is, furthermore, preferentially coupled with a process of affinity maturation.

Various mutation techniques, random or directed, well known in the state of the art, are used in order to increase the affinity of said antibodies. The latter mimic in vitro the process of affinity maturation. The antibodies thus produced are then selected, in particular using phage display.

The introduction of mutations by chain shuffling, as described in the documents Clakson et al., (1991); Marks et al., (1992); S. G. Park el al. (2000) is one of the first techniques which can be used. VL and VH domains with a high affinity are selected independently from different domain libraries, then fused. A selection then makes it possible to isolate the clone with higher affinity, said affinity being of the order of that of an antibody isolated during an in vivo secondary response. When it is not the entire domain which is mutated, the term “DNA shuffling” is used (Crameri et al., Nature medicine, 2(1):100-2 (1996).

An alternative technique can be error-prone PCR, described by Hawkins et al., 1992; Gram et al., 1992. This PCR is characterized by the use of a polymerase which induces far more error than does a standard enzyme during PCR amplification (on average 1.7 bases changed per variable domain). Numerous random mutations are thus inserted into the sequences. The database obtained is then selected using the antigen in order to select the clones with increased affinities.

Degeneration by directed mutagenesis also makes it possible to increase the affinity of the antibodies. It involves mutations in the amino acids situated in the hypervariable loops.

This technique can be applied to all the CDRs, one after the other, and the affinity gains can be additive (Barche et al., 1994; Balint et al., 1993). A variant of this technique called CDR walking consists of mutating antibodies only in a maximum of six CDRs (Yang et al., J. Mol. Biol. 254, 392-403 (1995). The document FR 2 924 431 describes obtaining antibodies with high affinity by said technique.

The use of mutagenic strains selected beforehand for their ability to induce a high level of somatic mutations in the variable domains of immunoglobulins is also a means of obtaining antibodies with a high affinity. The document Holliger et al., (1995) is an example of this. It describes the use of the mutD5 strain which induces point mutations, in particular along the scFv gene. The antibodies thus produced are then selected against the antigen, the stringency of the selections being increased at each selection round. The document S. J. Cumbers, et al., Nature Biotechnology, 20, 1129-1134 (2002) can also be used for reference.

The phage display technique using modified vectors as described in the document WO 2007/074496 or selection using phage display followed by that of Biopanning (Krebber et al., (1997); WO 2006/117699) is also another alternative to obtaining antibodies with high affinity.

Said technologies can also, for the purposes of the invention, be used with methods derived from phage display such as ribosome display (Irving, R. A et al., (2001)) or yeast display (Chao, G et al., J. Mol. Biol. 342(2):539-50, 2004).

Another alternative which is well known to a person skilled in the art is also the use of the MutaGen™ technique of molecular evolution, developed by Millegen, and described in the U.S. Pat. No. 7,670,809. This technology makes it possible to mimic somatic hypermutation, a natural phenomenon observed during the maturation of antibodies in vivo.

The Massive Mutagenesis® technology, developed by Biométhodes (EP 1311670) is also a possible solution. This is an intermediate technique between directed mutagenesis and random mutagenesis. The production of antibodies with high affinity obtained by said technology is described in the document WO 2009/050388.

These different technologies are not exhaustive.

The antibodies directed either against a circulating proinflammatory cytokine, or against a circulating bacterial toxin contained in the composition according to the invention have a high affinity for the FcγRIIIs. In particular, this high affinity can be associated with a small quantity of fucose on the glycan chains borne by the antibodies. This quantity of fucose, or fucose level, is defined as the average proportion of fucose borne by all the antibodies, with respect to the maximum quantity of fucose that the glycan chains can bear.

The fucose level can be defined in two ways:
either it is considered that all the antibodies of the composition are fucosylated in the same way, reasoning in terms of fucosylation in the case of one antibody, said antibody being representative of the composition,
or it is considered that each antibody is fucosylated differently, and the fucose level will be the average of the individual fucosylation of each antibody composing the composition of the invention.

In the first abovementioned case, if an antibody comprises one N-glycosylation site per heavy chain, and each glycosylation site is capable of binding a glycan chain bearing a fucose, said antibody will thus have the possibility of comprising a maximum of 2 fucoses.

Thus, the population of antibodies comprising on average one fucose will then have a fucose level of 50% (i.e. 1×100/2).

In the second abovementioned case, if the composition is composed of 10 antibodies, for example 3 antibodies are not fucosylated, 3 antibodies bear one fucose and 4 antibodies bear 2 fucoses (each antibody can contain up to 2 fucoses), the fucosylation level of the composition is 55%, i.e. 11 fucoses out of a possible 20.

The antibodies of the composition according to the invention can preferably be produced by clones derived from the cell lines such as Vero (ATCC No. CCL 81), YB2/0 (ATCC No. CRL 1662) or CHO Lec-1 (ATCC No. CRL 1735).

In an advantageous embodiment, the invention relates to a composition as defined previously, where each of the antibodies directed either against a circulating proinflammatory cytokine, or against a circulating bacterial toxin has, on the glycosylation site in position 297 of its heavy chains, one of the biantennary glycan forms selected from the following structures:

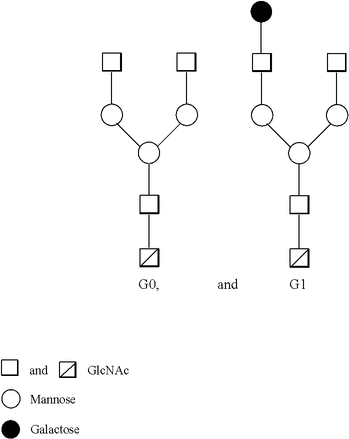

the GlcNAc represented by [symbol] in the above structures G0 and G1 being capable of being fucosylated.

The antibodies of the composition according to the invention bear, on the amino acid in position 297 of each of the heavy chains a particular glycan structure confering an effector activity dependent on FcγRIII.

Such antibodies can be obtained based on a method known to a person skilled in the art, as described in WO 01/77181 and have, on their glycosylation site (Asn 297), biantennary-type glycan structures, with short chains and low sialylation. Preferably, their glycan structure has terminal mannoses and/or non-intercalary terminal N-acetyl-glucosamine (GlcNAc).

The GlcNAc represented [symbol] in the structures G0 and G1 can therefore be either non-fucosylated, or bear one fucose molecule.

In another advantageous embodiment, the invention relates to a composition defined above, where each of the antibodies has, on the glycosylation site in position 297 of its heavy chains, one of the biantennary glycan forms selected from the following structures:

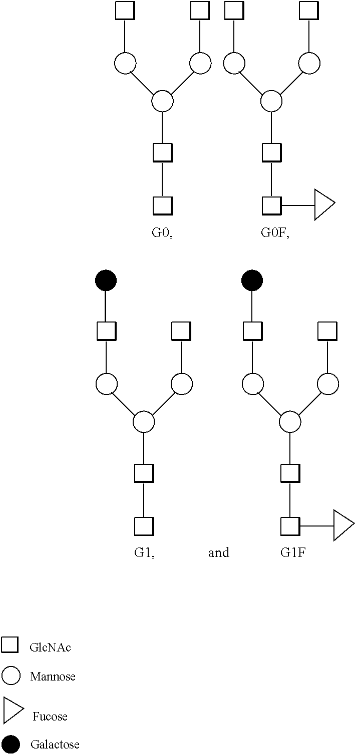

Thus, the antibodies contained in the composition of the invention are characterized in that they have on the glycosylation site Asn 297:
biantennary-type glycan structures, with short chains,
low sialylation,
non-intercalary terminal mannoses and/or GlcNAcs.

Advantageously, the antibodies comprised in the composition according to the invention have an average sialic acid content of less than 25%, 20%, 15%, or 10%, preferably 5%, 4%, 3% or 2%. The sialylation level is defined in the same way as the fucose level, as specified above.

In yet another advantageous embodiment, the invention relates to a composition described previously, characterized in that the G0F+G1F forms of the antibodies of said composition represent less than 50% of the glycan structures borne by the glycosylation site in position 297 of the heavy chain (Asn 297).

Thus, less than half of the antibodies contained in the composition according to the invention have, in position 297, a biantennary glycan chain bearing a fucose.

Even more advantageously, the fucose level is from approximately 20% to approximately 45%.

Another advantageous embodiment of the invention relates to a composition mentioned previously, where the G0+G1+G0F+G1F forms of the antibodies of said composition represent more than 60% of said glycan structures and preferably more than 80% of the glycan structures borne by the glycosylation site in position 297 of the heavy chain.

The present invention also relates to populations of antibodies directed either against a circulating proinflammatory cytokine, or against a circulating bacterial toxin, said antibodies having a high galactosylation level, and the compositions containing them.

The present invention also relates to a process for producing said antibodies directed either against a circulating proinflammatory cytokine, or against a circulating bacterial toxin, said antibodies having a high galactosylation level.

The present invention also relates to an antibody directed either against a circulating proinflammatory cytokine, or against a circulating bacterial toxin, the antibodies being highly galactosylated.

The present invention relates to a composition defined above, in which the galactosylation level of all of the antibodies of the population is at least 60%.

The present invention relates to a composition defined above, in which the galactosylation level of all of the antibodies of the population is at least 70%.

The present invention relates to a composition defined above, in which the galactosylation level of all of the antibodies of the population is at least 80%.

The present invention relates to a composition defined above, in which the fucosylation level of all of the antibodies of the population is at least 50%.

The present invention relates to a composition defined above, in which the fucosylation level of all of the antibodies of the population is at least 60%.

The present invention relates to a composition defined above, in which the population comprises antibodies which comprise mono-galactosylated N-glycans.

The present invention relates to a composition defined above, in which the population comprises antibodies which comprise bi-galactosylated N-glycans.

The present invention relates to a composition defined above, in which the ratio of the galactosylation level of the antibodies of the population to the fucosylation level of the antibodies of the population is comprised from 1.0 to 1.4.

The present invention relates to a composition defined above, in which at least 35% of the antibodies in the population comprise bi-galactosylated N-glycans and at least 25% of the antibodies in the population comprise mono-galactosylated N-glycans.

The present invention relates to a composition defined above, in which the antibody is produced in the mammary epithelial cells of a non-human mammal.

The present invention relates to a composition defined above, in which the antibody is produced in a transgenic non-human mammal, in particular in a goat, a sheep, a bison, a camel, a cow, a pig, a rabbit, a buffalo, a horse, a rat, a mouse or a llama.

The present invention relates to a composition defined above, also comprising milk.

The present invention relates to a composition defined above, also comprising a pharmaceutically acceptable vehicle.

The present invention also relates to populations of antibodies with a high mannosylation level, and the compositions containing them.

The present invention also relates to a process for producing said antibodies with a high mannosylation level.

The present invention also relates to an antibody, the antibody being highly mannosylated.

Populations of Antibodies Directed Either Against a Circulating Proinflammatory Cytokine. Or Against a Circulating Bacterial Toxin

The antibodies according to the invention bear an oligosaccharide chain at each asparagine 297 (Asn297, Kabat numbering) of each of the heavy chains.

It is well known to a person skilled in the art that antibodies having oligosaccharide chains containing little or no fucose, have a better affinity for the CD16 (FcγRIIIa) present on the effector cells of the immune system.

Thus, a composition of antibodies having a fucose content of less than 65% at the level of the oligosaccharide chains borne by Asn297 is particularly preferred. The fucose content can be measured by well known methods, for example by the MALDI-TOF, HPCE-LIF, or HPLC method.

Advantageously, the invention relates to a composition as defined above in which the fucose level of the antibodies, or the fucose content of the antibodies, is less than 60%, or less than 50%, or less than 40%, or at least 30%, or even equal to 0%. It being understood that a fucose content equal to 0% corresponds to 100% of the oligosaccharide chains borne by Asn297 which are without fucose. Alternatively, the fucose content can be comprised between 0% and 50% or between 10% and 50% or between 20 and 50%.

In a particular embodiment, the antibodies according to the invention can also comprise oligosaccharide chains which have a bisection.

By “bisection” is meant, within the meaning of the present invention, any intercalary N-acetylglucosamine residue grafted to β1.4 (intercalary GlcNac), in particular by the action of β1,4-N-Acetylglucosaminyltransferase III (GnTIII).

In a particular embodiment, a composition of antibodies according to the invention has a bisection content of at least 20%, for example at least 30%, or at least 40%, or also at least 50%, 60%, 70%.

Methods for producing antibodies of the invention comprising at least one Fc domain of an immunoglobulin and having intercalary N-acetylglucosamine residues (intercalary GlcNac) are for example described in the documents EP 1 071 700 and U.S. Pat. No. 6,602,684 or EP 1 692 182 and US 2005/123546, this list not being limitative. For example, the antibodies according to the invention can be produced in a host cell expressing at least one nucleic acid coding for a polypeptide having a β-(1,4)-N-acetylglucosaminyltransferase III activity in a quantity sufficient to modify the glycosylation borne by the Fc domain(s) of said antibodies.

In another embodiment, the antibodies according to the invention have low fucosylation, i.e. glycan structures having a fucose content of less than 65%. In a particular embodiment, the antibodies according to the invention have glycan structures having a content of less than 50% in the case of the G0F+G1F forms. In a particular embodiment, the antibodies comprise a content greater than 60% in the case of the G0+G1+G0F+G1F forms, that of the G0F+G 1F forms being less than 50%. In another particular embodiment, the antibodies comprise a content greater than 60% in the case of the G0+G1+G0F+G1F forms, the fucose content being less than 65%. These forms are selected from the G0, G0F, G1 and GIF forms, as described in the present Application.

In this other embodiment, the antibodies also comprise, on the Asn297 glycosylation sites, a glycan structure having terminal mannoses and/or non-intercalary terminal N-acetylglucosamines.

In a particular embodiment, the antibodies comprise, on the Asn297 glycosylation site, a glycan structure of biantennary type, with short chains, low sialylation, and a content greater than 60% in the case of the G0+G1+G0F+G1F forms, that of the G0F+G1F forms being less than 50%.

In particular, the antibodies have a sialic acid content of less than 25%, 20%, 15%, or 100%, preferably 5%, 4% 3% or 2%.

In a particular embodiment, the antibodies according to the invention have glycan structures as described in WO 01/77181. The antibodies can in particular be selected by selecting a cell line capable of producing antibodies having a high affinity for the CD16 receptor. For example, the antibodies may be produced in a hybridoma, in particular a heterohybridoma obtained with the fusion partner K6H6-B5 (ATCC CRL 1823) or in an animal or human cell producing said antibodies, in particular a cell derived from the Vero lines (ATCC CCL-81), a rat hybridoma cell line, such as for example the rat hybridoma line YB2/0 (ATCC CRL-1662, YB2/3HL.P2.G1.16Ag.20 cell, deposited at the American Type Culture Collection) or also the CHO line Lec-1 (ATCC CRL-1735), CHO-Lec10, CHO dhfr- (for example CHO DX BII, CHO DG44), CHO Lec13, SP2/0, NSO, 293, BHK, COS, IR983F, a human myeloma such as Namalwa or any other cell of human origin such as PERC6, CHO Pro-5, CHO dhfr- (CHO DX BII, CHO DG44), Wi1-2, Jurkat, Vero, Molt-4, COS-7, 293-HEK, BHK, K6H6, NSO, SP2/0-Ag 14 and P3X63Ag8.653.

Advantageously, the antibodies can be produced in a cell line selected from YB2/0, Vero, CHO-lec10, CHO-lec13, CHO-lec1, CHOK1SV, CHO Knock Out in the case of FUT8 fucosyltransferase, CHO expressing the GnTIII.

Particularly advantageously, the antibodies of the invention are produced in a rat hybridoma cell line. In a preferred embodiment, the antibodies are produced in the rat hybridoma YB2/0 (YB2/3HL.P2.G11.16Ag.20 cell, deposited at the American Type Culture Collection under number ATCC CRL-1662) selected for its ability to produce antibodies having a high affinity for CD16.

Other methods are known to a person skilled in the art, for producing antibodies with optimized glycosylation. For example the use of a glycosylation inhibitor such as kifunensine (alpha mannosidase 11 inhibitor), which can be added to the culture medium of the cells producing antibodies according to the invention, according to the method described in U.S. Pat. No. 7,700,321. Fucose analogues can also be introduced into the culture medium of cells producing antibodies as described in the document US 20090317869.

Another means for producing antibodies with optimized glycosylation is the use of cells for which the GDP-fucose production pathway is inhibited, via the inhibition of at least one of the enzymes of the fucose production cycle, as described for example in the document US 2010291628 or US 20090228994, the document EP 1 500 698, the document EP 1 792 987 or also the document U.S. Pat. No. 7,846,725, this list not being limitative. It is also possible to use RNA interference (RNAi) inhibiting 1,6-fucosyltransferase as described in the document U.S. Pat. No. 7,393,683 or the document WO 2006133148.

Other methods for preparing antibodies with optimized glycosylation in transgenic animals are described in WO 200748077. The antibodies can also be produced in yeasts, as shown in the document WO 0200879.

In order to produce antibodies with 100% non-fucosylated oligosaccharides, i.e. totally devoid of fucose at the glycosylation level borne by the Asn297, it is possible to implement the preparation methods described in the documents EP 1 176 195, U.S. Pat. Nos. 7,214,775, 6,994,292, 7,425,446, US2010223686, WO2007099988, EP 1 705 251, this list not being limitative. In a particular embodiment, the antibodies are produced in cells modified by deletion of the gene coding for α1,6-fucosyltransferase or by adding a mutation of this gene in order to eliminate the α1,6-fucosyltransferase activity.

According to another aspect, the invention relates to a composition comprising a population of antibodies directed either against a circulating proinflammatory cytokine, or against a circulating bacterial toxin, and in which the galactosylation level of the antibodies of the population is at least 60%.

According to yet another aspect, the galactosylation level of the antibodies of the population is at least 70%.

According to yet another aspect, the galactosylation level of the antibodies of the population is at least 80%.

According to yet another particular aspect, the fucosylation level of all of the antibodies of the population is at least 50%, and in particular at least 60%.

According to another particular aspect, the population comprises antibodies which comprise mono-galactosylated N-glycans.

According to another particular aspect, the population comprises antibodies which comprise bi-galactosylated N-glycans.

According to another particular aspect, the ratio of the galactosylation level of the antibodies of the population to the fucosylation level of the antibodies of the population is from 1.0 to 1.4.

According to another particular aspect, at least 35% of the antibodies in the population comprises bi-galactosylated N-glycans and at least 25% of the antibodies in the population comprises mono-galactosylated N-glycans.

According to another particular aspect, the sialylation level of the antibodies is at least 50%.

According to yet another particular aspect, the sialylation level of the antibodies is at least 70%.

According to yet another particular aspect, the sialylation level of the antibodies is at least 90%.

According to yet another particular aspect, the antibodies are totally sialylated.

The biosynthesis of the N-glycans is not regulated by coding, as is the case with the proteins, but is mainly dependent on the expression and activity of the specific glycosyltransferases in a cell. Thus, a glycoprotein, such as the Fc fragment of an antibody, normally exists as a heterogeneous population of glycoforms which bear different glycans on the same protein backbone.

A population of highly galactosylated antibodies is a population of antibodies in which the galactosylation level of all of the antibodies of the population is at least 50%, at least 60%, at least 70%, at least 80%, at least 90%, up to 100% galactosylation.

According to a particular embodiment of the population of highly galactosylated antibodies, the galactosylation level of all of the antibodies of the population is at least 60%.

The galactosylation level can be determined with the following formula:

$$\frac{{\sum }_{i = 1}^{n} ( number of Gal ) * ( \% of relative surface area )}{{\sum }_{i = 1}^{n} ( number of A ) * ( \% of relative surface area )} * 100$$

in which:
“n” represents the number of N-glycan peaks analyzed on a chromatogram, for example of a normal phase high performance liquid chromatography (NP HPLC) spectrum,
“number of Gal” represents the number of galactoses on the antenna of the glycan corresponding to the peak,
“number of A” represents the number of N-acetyl-glucosamine antennae of the glycan form corresponding to the peak, and
“% of relative surface area” corresponds to the percentage of the area under the corresponding peak.

The galactosylation level of the antibodies of the population of antibodies can be determined, for example, by releasing the N-glycans from the antibodies, by resolving the N-glycans on a chromatogram, by identifying the oligosaccharide unit of the N-glycan which corresponds to a specific peak, by determining the intensity of the peak and applying the data to the abovementioned formula.

Antibodies which are galactosylated include antibodies which have mono-galactosylated N-glycans and bi-galactosylated N-glycans.

According to a particular aspect of the population of highly galactosylated antibodies, the population comprises antibodies which comprise mono-galactosylated N-glycans, which may or may not be sialylated. According to a particular aspect of the population of highly galactosylated antibodies, at least 1%, at least 5%, at least 100%, at least 15%, at least 20%, at least 25%, at least 30%, at least 40%, at least 50%, at least 60%, at least 70%, at least 80%, at least 90%, up to 100% of the N-glycans of the antibodies comprise mono-galactosylated N-glycans. According to another particular embodiment of the invention, in the population of highly galactosylated antibodies, at least 25% of the antibodies comprise mono-galactosylated N-glycans.

According to a particular aspect of the population of highly galactosylated antibodies, the population comprises antibodies which comprise bi-galactosylated N-glycans, which may or may not be sialylated. According to a particular aspect of the population of highly galactosylated antibodies, at least 1%, at least 5%, at least 10%, at least 15%, at least 20%, at least 25%, at least 30%, at least 40%, at least 50%, at least 60%, at least 70%, at least 80%, at least 90%, up to 100% of the N-glycans of the antibodies comprise bi-galactosylated N-glycans. According to another particular embodiment of the invention, in the population of highly galactosylated antibodies, at least 35% of the antibodies comprise bi-galactosylated N-glycans.

According to yet another aspect of the population of highly galactosylated antibodies, the population comprises antibodies which comprise mono-galactosylated N-glycans, which may or may not be sialylated, and antibodies which comprise bi-galactosylated N-glycans, which may or may not be sialylated.

According to a particular aspect of the population of highly galactosylated antibodies, at least 1%, at least 5%, at least 10%, at least 15%, at least 20%, at least 25%, at least 30%, at least 40%, at least 50%, at least 60%, at least 70%, at least 80%, at least 90%, up to 99% of the N-glycans of the antibodies comprise mono-galactosylated N-glycans, and at least 1%, at least 5%, at least 10%, at least 15%, at least 20%, at least 25%, at least 30%, at least 40%, at least 50%, at least 60%, at least 70%, at least 80%, at least 90%, up to 99% of the N-glycans of the antibodies comprise bi-galactosylated N-glycans.

According to another particular aspect of the population of highly galactosylated antibodies, at least 25% of the antibodies comprise mono-galactosylated N-glycans, and at least 35% of the antibodies comprise bi-galactosylated N-glycans.

According to yet another aspect of the population of highly galactosylated antibodies, the population comprises highly fucosylated antibodies. A population of highly fucosylated antibodies is a population of antibodies in which the fucosylation level of the N-glycans of the antibodies of the population is at least 40%, at least 50%, at least 60%, at least 70%, at least 80%, at least 90%, up to 100% fucosylation. According to a particular aspect of the population of highly galactosylated antibodies, the fucosylation level of the N-glycans of the antibodies is at least 50%.

The fucosylation level can be determined using the following formula:

$${\sum }_{i = 1}^{n} ( number of Fucoeses ) * ( \% of relative surface area )$$

in which:
“n” represents the number of N-glycan peaks analyzed on a chromatogram, for example of a normal-phase high performance liquid chromatography (NP HPLC) spectrum,
“number of Fucoses” represents the number of fucoses on the glycan structure corresponding to the peak,
“% of relative surface area” corresponds to the percentage of the area under the corresponding peak containing the fucose.

Antibodies which are fucosylated include antibodies which have at least one fucose monosaccharide on one of their N-glycans. The antibodies which are fucosylated include antibodies which have at least one fucose monosaccharide on each of their N-glycans.

According to a particular aspect, the population of antibodies refers to a population in which the galactosylation level of the N-glycans of the antibodies in the population is at least 60%, and the fucosylation level of the antibodies in the population is at least 50%.

According to a particular aspect, the population of antibodies refers to a population in which the galactosylation level of the N-glycans of the antibodies in the population is at least 50%, and the fucosylation level of the N-glycans of the antibodies in the population is at least 50%, at least 60%, at least 70%, at least 80%, at least 90%, up to 100%.

According to yet another particular aspect, the population of antibodies described here refers to a population in which the galactosylation level of the N-glycans of the antibodies in the population is at least 60%, and the fucosylation level of the N-glycans of the antibodies in the population is at least 50%, at least 60%, at least 70%, at least 80%, at least 90%, up to 100%.

According to yet another particular aspect, the population of antibodies described here refers to a population in which the galactosylation level of the N-glycans of the antibodies in the population is at least 70%, and the fucosylation level of the N-glycans of the antibodies in the population is at least 50%, at least 60%, at least 70%, at least 80%, at least 90%, up to 100%.

According to yet another particular aspect, the population of antibodies described here refers to a population in which the galactosylation level of the N-glycans of the antibodies in the population is at least 80%, and the fucosylation level of the N-glycans of the antibodies in the population is at least 60%, at least 70%, at least 80%, at least 90%, up to 100%.

According to yet another particular aspect, the population of antibodies described here refers to a population in which the galactosylation level of the N-glycans of the antibodies in the population is at least 90%, and the fucosylation level of the N-glycans of the antibodies in the population is at least 60%, at least 70%, at least 80%, at least 90%, up to 100%.

According to yet another particular aspect, the population of antibodies described here refers to a population in which the galactosylation level of the N-glycans of the antibodies in the population is up to 100%, and the fucosylation level of the N-glycans of the antibodies in the population is at least 60%, at least 70%, at least 80%, at least 90%, up to 100%.

According to yet another aspect of the invention, the invention relates to a composition comprising a population of antibodies directed either against a circulating proinflammatory cytokine, or against a circulating bacterial toxin with a specific ratio between the percentages of the N-glycans of the antibodies in the population which are galactosylated on the galactosylation site of the Fc gamma fragment and the percentages of the N-glycans of the antibodies in the population which are fucosylated on the glycosylation site of the Fc gamma fragment.

According to a particular aspect, the invention relates to a composition comprising a population of antibodies directed either against a circulating proinflammatory cytokine, or against a circulating bacterial toxin in which the ratio between the galactosylation level of the N-glycans of the antibodies in the population and the fucosylation level of the N-glycans of the antibodies in the population is between 0.5 and 2.5; between 0.6 and 2.0; between 0.7 and 1.8 or between 1.0 and 1.4.

According to yet another particular aspect, the invention relates to a composition comprising a population of antibodies in which the ratio between the galactosylation level of the N-glycans of the antibodies in the population and the fucosylation level of the N-glycans of the antibodies in the population is between 1.0 and 1.4, for example 1.2.

According to yet another aspect of the invention, the antibodies of the invention directed either against a circulating proinflammatory cytokine, or against a circulating bacterial toxin have been modified in order to contain an oligomannose or an additional oligomannose. In another embodiment, the antibodies have been modified so that at least 30% of the antibodies contain at least one oligomannose. In another embodiment, the antibodies have been modified so that at least 40%, 50%, 60%, 70%, 80%, 90% of the antibodies contain at least one oligomannose. In another embodiment, the antibodies have been modified so that less than 50%, 40%, 30%, 20%, 10%, of the antibodies contain fucose on at least one antibody chain. In yet another embodiment, the antibodies have been modified so that at least 40%, 50%, 60%, 70%, 80%, 90% of the antibodies contain at least one oligomannose and less than 50%, 40%, 30%, 20%, 10%, of the antibodies contain fucose on at least one antibody chain.

In another embodiment, the N-glycans of the antibodies directed either against a circulating proinflammatory cytokine, or against a circulating bacterial toxin have been modified in order to have a highly mannosylated glycosylation profile. In yet another embodiment, the antibodies have been modified so that at least one antibody chain contains an oligomannose and is non-fucosylated. In yet another embodiment, the antibodies have been modified so that the major N-glycan of the antibodies is non-fucosylated. In an embodiment the main carbohydrate is a non-fucosylated oligomannose. In another embodiment, the main N-glycan is a non-fucosylated Man5. In yet another embodiment, the antibodies have been modified so that less than 40% of the carbohydrates of the antibodies contain fucose. In yet another embodiment, the antibodies have been modified so that less than 30%, 20%, 10% of the carbohydrates of the antibodies contain fucose. In an embodiment the fucose is 1,6-fucose. In another embodiment, the antibodies have been modified so that at least 60% of the N-glycans of the antibodies are a non-fucosylated oligomannose and less than 40% of the carbohydrates are antibodies containing fucose.

In yet another embodiment, the antibodies are modified so that the N-glycans of the antibodies have a highly mannosylated glycosylation profile. In an embodiment the antibodies are modified so that at least one antibody chain contains an oligomannose and is non-fucosylated. In another embodiment, the antibodies are modified so that one antibody chain contains an oligomannose and is non-fucosylated. In another embodiment, the antibodies are modified so that the major N-glycan of the antibodies is non-fucosylated. In an embodiment the main N-glycan is a non-fucosylated oligomannose. In another embodiment, the main N-glycan is a non-fucosylated Man5. In yet another embodiment, the antibodies are modified so that less than 40% of the N-glycans of the antibodies contain fucose. In an embodiment the antibodies are modified so that less than 30%, 20%, 10% or less of the carbohydrates contain fucose antibodies. In yet another embodiment, the antibodies are modified so that at least 30% of the antibodies have at least one oligomannose. In an embodiment the antibodies are modified so that at least 40%, 50%, 60%, 70%, 80%, 90% of the antibodies have at least one oligomannose. In yet another embodiment, the antibodies are modified so that at least 40%, 50%, 60%, 70%, 80%, 90% of the antibodies contain at least one oligomannose and less than 50%, 40%, 30%, 20%, 10%, of the antibodies contain a fucose on at least one chain. In another embodiment, the antibodies are modified so that at least 60% of the N-glycans of the antibodies are a non-fucosylated oligomannose and less than 40% of the N-glycans of the antibodies contain fucose.

The expression “highly mannosylated glycosylation profile” is intended to denote an antibody which contains at least one oligomannose or a composition of antibodies in which at least 30% of the antibodies contain at least one oligomannose. In certain embodiments at least 30%, 40%, 50%, 60%, 70%, 80%, 90% of the N-glycans of the antibodies are an oligomannose. In certain embodiments at least 30%, 40%, 50%, 60%, 70%, 80%, 90% of the N-glycans of the antibodies are a non-fucosylated oligomannose. In other embodiments less than 50%, 40%, 30%, 20%, 10%, 5% of the N-glycans of the antibodies contain fucose. In other embodiments, the antibodies are low in fucose and rich in oligomannose.

As a result, in other embodiments, at least 30%, 40%, 50%, 60%, 70%, 80% or 90% of the N-glycans of the antibodies are an oligomannose and less than 50%, 40%, 30%, 20%, 10% or 5% of the N-glycans of the antibodies contain fucose. As a result, in yet another embodiment, at least 30%, 40%, 50%, 60%, 70%, 80% or 90% or more of the N-glycans of the antibodies are non-fucosylated oligomannose and less than 50%, 40%, 30%, 20%/0, 10% or 5% of the N-glycans are antibodies containing fucose. An embodiment of the invention relates to antibodies binding to the Fc gamma RIII receptor found on the monocytes, macrophages and enhanced natural killer cells, which do not have a 1,6-fucose sugar on the heavy chain.

The present invention also relates to a composition of antibodies directed either against a circulating proinflammatory cytokine, or against a circulating bacterial toxin in which the antibodies contain an oligommanose.

The present invention also relates to a composition in which at least 30% of the antibodies contain at least one oligomannose.

The present invention also relates to a composition in which the N-glycans of the antibodies have a highly mannosylated glycosylation profile.

The present invention also relates to a composition in which at least one chain of the antibodies contains an oligomannose and is not fucosylated.

The present invention also relates to a composition in which the major N-glycan of the antibodies is not fucosylated.

The present invention also relates to a composition in which the major N-glycan of the antibodies is a non-fucosylated oligomannose.

The present invention also relates to a composition in which the major N-glycan of the antibodies is a non-fucosylated Man5.

The present invention also relates to a composition in which less than 40% of the N-glycans of the antibodies contain fucose.

The present invention also relates to a composition in which the antibodies contain no fucose.

The present invention also relates to a composition in which at least 60% of the N-glycans of the antibodies contain a fucosylated oligomannose and in which less than 40% of the N-glycans of the antibodies contain fucose.

The antibodies according to the invention can be produced by the techniques described in the international application WO/2007/048077 or also in the American provisional application 61,065 13 Feb. 2013 which are incorporated here by way of reference.

The profiles of the antibodies described in the international application WO/2007/048077 or also in the American provisional application 61,065 of 13 Feb. 2013 are also incorporated by way of reference.

Advantageously, the antibodies have a high level of complement-dependent cytotoxicity (CDC) activity and/or a high level of antibody-dependent cellular cytotoxicity (ADCC) activity.

The invention is better illustrated by the following examples and figures. The examples below are intended to clarify the subject-matter of the invention and illustrate advantageous embodiments, but are in no way intended to restrict the scope of the invention.

### CAPTIONS TO THE FIGURES

FIG. 1: Chromatogram representing the N-glycans of a population of highly galactosylated adalimumab antibodies originating from goat #1.

FIG. 2: Chromatogram representing the N-glycans of a population of highly galactosylated adalimumab antibodies originating from goat #1 at 7 days of lactation.

FIG. 3: Chromatogram representing the N-glycans of a population of highly galactosylated adalimumab antibodies originating from goat #1 at 17 days of lactation.

FIG. 4: Chromatogram representing the N-glycans of a population of highly galactosylated adalimumab antibodies originating from goat #1 at 32 days of lactation.

FIG. 5: Summary of the percentages of the oligosaccharides of the N-glycans of the populations of highly galactosylated adalimumab antibodies originating from goat #1 at various days of lactation.

FIG. 6: Chromatogram representing the N-glycans of a population of highly galactosylated adalimumab antibodies originating from goat #2 at 3 days of lactation.

FIG. 7: Chromatogram representing the N-glycans of a population of highly galactosylated adalimumab antibodies originating from goat #2 at 11 days of lactation.

FIG. 8: Chromatogram representing the N-glycans of a population of highly galactosylated adalimumab antibodies originating from goat #2 at 21 days of lactation.

FIG. 9: Summary of the percentages of the oligosaccharides of the N-glycans of the populations of highly galactosylated adalimumab antibodies originating from goat #2 at various days of lactation.

FIG. 10: Transgenically produced adalimumab antibodies binding to the soluble TNF-α.

FIG. 11: Transgenically produced adalimumab antibodies binding to the CD16 expressed on the NK cells after competition with an anti-CD16 3G8 antibody.

FIG. 12 corresponds to the representation of the ability to induce phagocytosis of the soluble TNF by the CD16+ macrophages.

### EXAMPLES

### Materials and Methods of Examples 1 and 2

### Production of Transgenic Goats Producing Adalimumab

Transgenic goats were produced by introducing into their genome the nucleic acid sequence coding for the adalimumab antibody. The goats producing the adalimumab were produced using conventional micro-injection techniques (cf. U.S. Pat. No. 7,928,064). The cDNA coding for the heavy and light chain (SEQ ID NO: 3 and SEQ ID NO: 4) is synthesized on the basis of the published amino acid sequence (U.S. Pat. No. 6,090,382). These DNA sequences were ligated with the beta casein expression vector in order to produce the BC2601 HC and LC BC2602 constructions. In these plasmids, the nucleic acid sequence coding for the adalimumab is under the control of a promoter facilitating the expression of adalimumab in the goats' mammary glands. The prokaryotic sequences were removed and the DNA was micro-injected into pre-implantation goat embryos. These embryos were then transferred to pseudogravid females. The resultant offspring were screened for the presence of the transgenes. Those bearing both chains were identified as transgenic founders.

The founder animals were raised to the appropriate age. After pregnancy and parturition, they were fed with milk. Lactation kinetics were realized in terms of days starting from parturition (for example, day 3, day 7, day 11). The adalimumab antibody was purified from the milk at each point in time and characterized as described here.

### Measurement of the Binding of the Transgenically Produced Adalimumab by the ELISA Method:

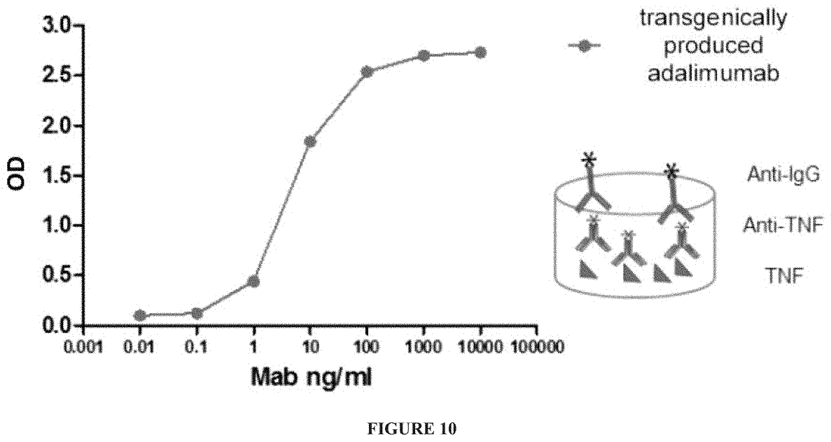

The TNF-α was coated overnight at 4° C. in a 96-well plate under 100 μl of PBS at a concentration of 5 μg/ml. After blocking the aspecific sites (incubation with 200 μl of PBS/BSA at 1%, 1 h at ambient temperature), the transgenically produced adalimumab or deglycosylated adalimumab was added at various concentrations (from 0 to 10 μg/ml) for 20 minutes in PBS/BSA at 1%. After washing, the binding of the transgenically produced adalimumab to the TNF-α was evaluated by the addition of goat anti-human IgG antibody (H+L) coupled with peroxidase, followed by the substrate (H₂O₂ and tetramethylbenzidine). After incubation for 20 minutes, the reaction was stopped with 50 μl of dilute H₂SO₄, and the OD was read at 450 nm. The results for the transgenically produced adalimumab are shown in FIG. 10.

Binding to the CD16, Competition with the 3G8 Antibody

In order to evaluate the binding of the transgenically produced adalimumab to the CD16, a binding displacement study with the anti-CD16 antibody 3G8, (Santa Cruz Biotech) was carried out. The displacement test made it possible to determine the efficiency of the binding of the transgenically produced adalimumab to the CD16 receptor expressed at the surface of the membrane of NK cells.

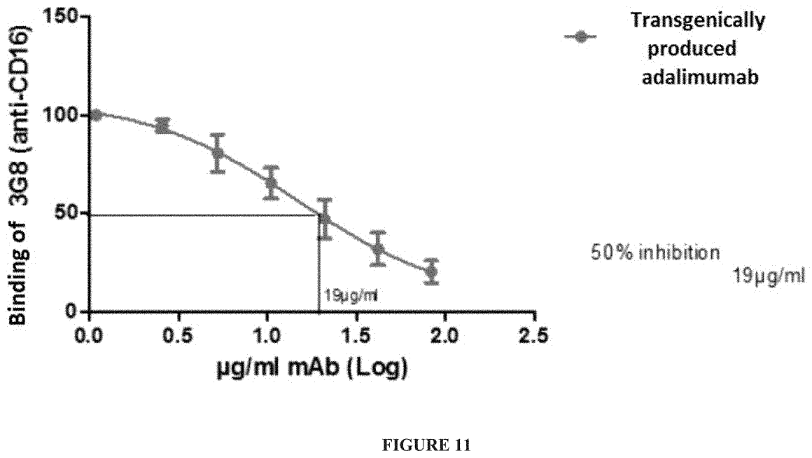

Natural killer cells (NK cells) were purified by negative depletion (Miltenyi) from the peripheral blood of healthy donors. The NK cells were then incubated at variable concentrations of the transgenically produced adalimumab (from 0 to 83 μg/ml) and at a fixed concentration of the anti-CD16 antibody 3G8 conjugated to a fluorochrome (3G8-PE). After washing, the binding of 3G8-PE to the CD16 receptor on the NK cells was evaluated by flow cytometry. The mean fluorescence values (MFVs) observed were expressed in binding percentages; a value of 100% corresponds to the value observed without the transgenically produced adalimumab and which therefore corresponds to the maximum binding of 3G8. A value of 0% corresponds to the MFV in the absence of antibody 3G8. The IC₅₀, i.e. the antibody concentration necessary to induce inhibition of 50% of the Imax of the binding of 3G8, was calculated using the PRISM software. The results are shown in FIG. 11.

Binding of Soluble TNF-α with the Transgenically Produced Adalimumab to the CD16 Expressed on the Jurkat Cells Via the Fc Fragment of the Transgenically Produced Adalimumab

Jurkat-CD16 cells were incubated with 10 μg/ml of transgenically produced adalimumab or the deglycosylated version of the latter for 20 minutes at 4° C. After washing, 100 μl of TNF-α was added to the cell pellet at a final concentration of 1 μg/ml, for 20 min at 4° C. After an additional washing, the cells were incubated with 5 μg/ml of a biotinylated goat anti-human TNF-α antibody, for 20 min at 4° C. After another washing cycle, the binding of the TNF-α was visualized by the addition of streptavidin coupled with PE fluorochrome for 20 min at 4° C. The samples were analyzed by flow cytometry.

### Results:

### Example 1: Transgenically Produced Adalimumab

The glycosylation profile of the adalimumab antibodies produced in the milk of transgenic goats was determined by the release of the N-glycans from the antibodies and column analysis of the oligosaccharides thus released

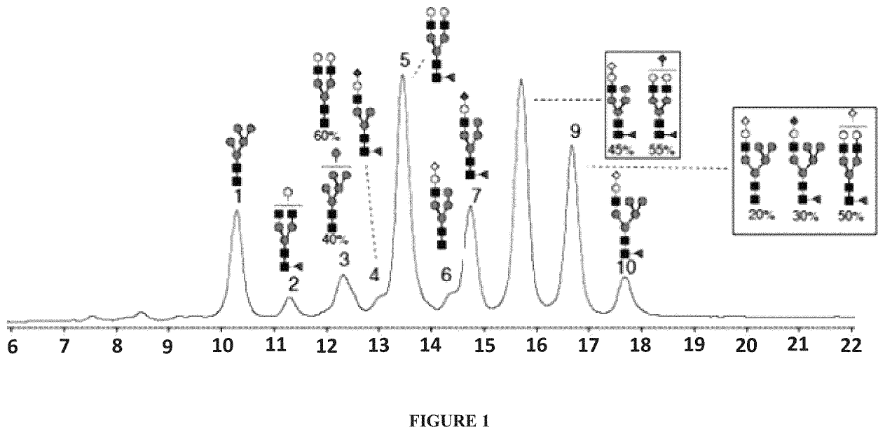

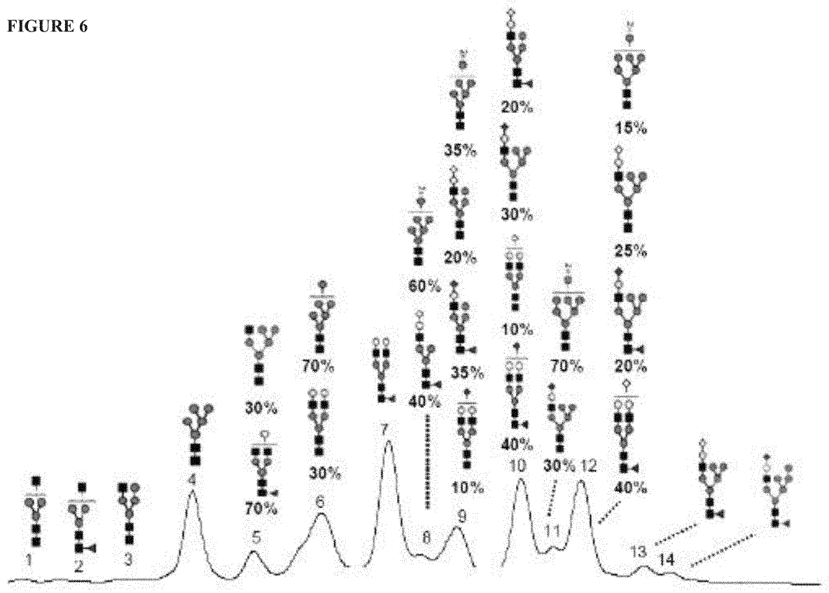

FIGS. 1-4 and 6-8 show the oligosaccharides (N-glycans) released from the transgenically produced adalimumab antibody originating from goat #1 (FIGS. 1-4) and from goat #2 (FIGS. 6-8). The groups of monosaccharides are represented as follows:
Black square: N-acetylglucosamine (GlcNAc)
Triangle: fucose
Grey circle: mannose
White circle: galactose
Grey diamond: N-glycolylneuraminic acid (NGNA): a sialic acid
White diamond: N-acetylneuraminic acid (NANA): a sialic acid

FIG. 1 shows a chromatogram representing the oligosaccharides (N-glycans) released from the transgenically produced adalimumab antibody in the milk of goat #1. The chromatogram shows that among the fourteen main oligosaccharides (N-glycans) produced, twelve have at least one galactose in the N-glycan chain, of which four oligosaccharides have two galactoses. Only two of the oligosaccharides are pure oligomannoses (See peak 1 and peak 3). FIG. 1 also shows that among the fourteen main oligosaccharides produced, nine are fucosylated. All the fucosylated oligosaccharides are also galactosylated.

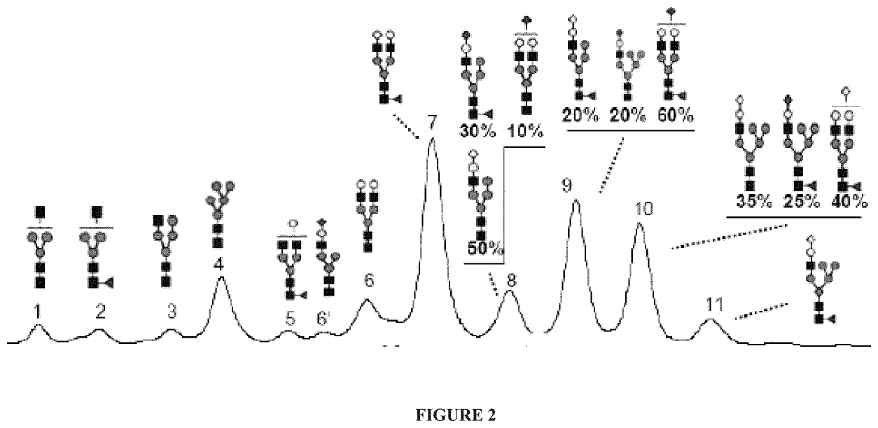

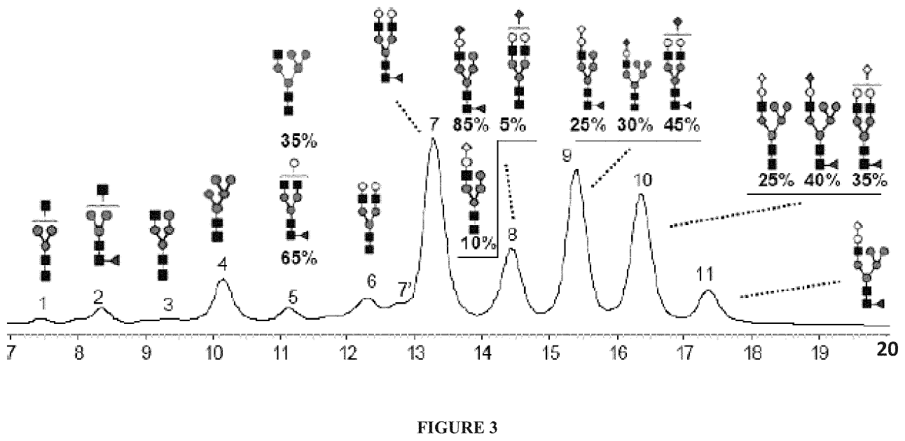

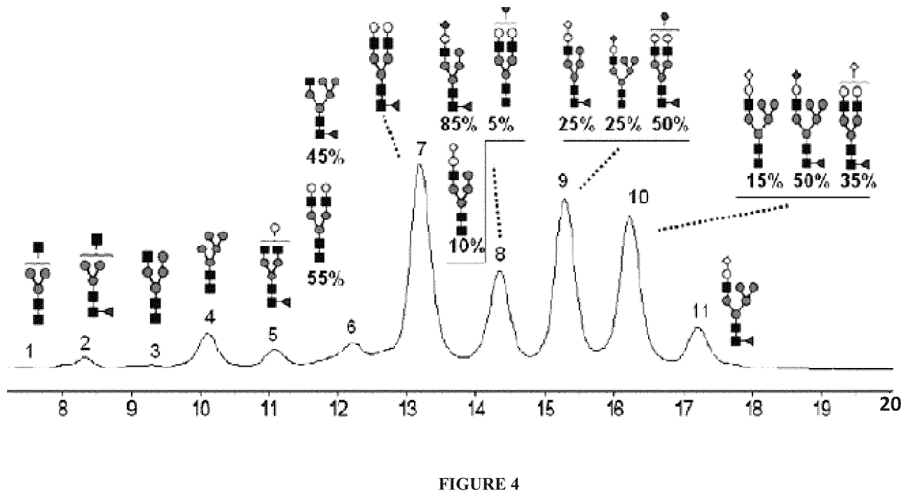

FIGS. 2-4 show the chromatograms of oligosaccharides (N-glycans) released from the transgenically produced adalimumab antibody in the milk of goat #1 as collected after 7 days of lactation (FIG. 2), 17 days of lactation (FIG. 3), and 32 days of lactation (FIG. 4).

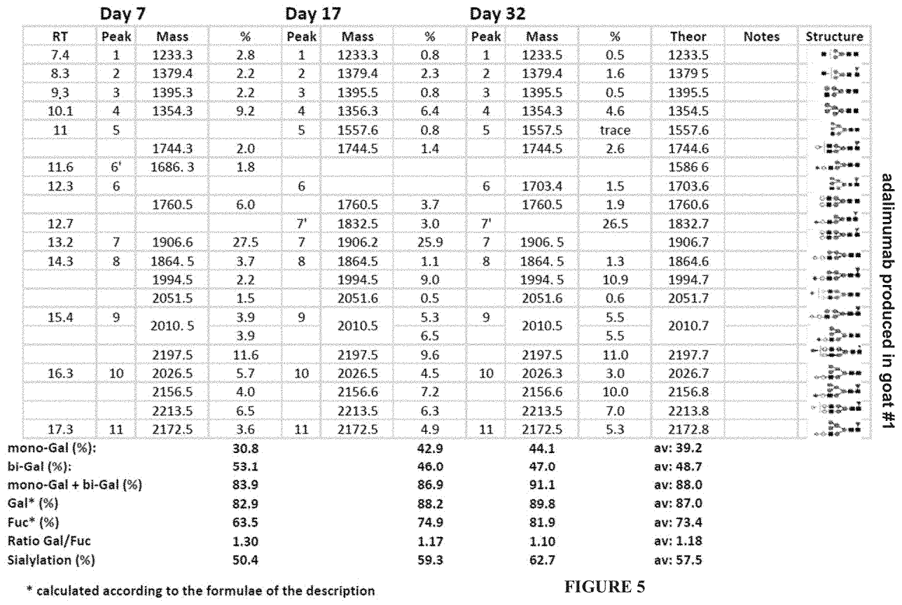

The relative percentages of all of the N-glycans isolated from the adalimumab antibody produced in the milk of goat #1 are illustrated in FIG. 5. FIG. 5 also shows a table of the overall percentage of mono-galactosylation, the percentage of bi-galactosylation, the total percentage of galactosylation (mono-galactosylation+bi-galactosylation), the percentage of galactosylation was calculated according to the formula indicated above, the percentage of fucosylation was calculated according to the formula indicated above, from the ratio of galactosylation to fucosylation and from the percentage of glycan structures having at least one sialic acid (% of sialylation). The results are also summarized in Table 1 below:

TABLE 1
oligosaccharides (N-glycans) isolated from the adalimumab antibodies
of goat #1

| | day 7 | day 17 | day 32 | average |
|---|---|---|---|---|
| mono-Gal (%): | 30.8 | 42.9 | 44.1 | 39.2 |
| bi-Gal (%): | 53.1 | 46.0 | 47.0 | 48.7 |
| mono-Gal + bi-Gal (%) | 83.9 | 88.9 | 91.1 | 88.0 |
| Gal* (%) | 82.9 | 88.2 | 89.8 | 87.0 |
| Fuc* (%) | 63.5 | 74.9 | 81.9 | 73.4 |
| Gal/Fuc ratio | 1.30 | 1.17 | 1.10 | 1.18 |
| Silaylation (%) | 50.4 | 59.3 | 62.7 | 57.5 |

*calculated according to the formulae given in the description

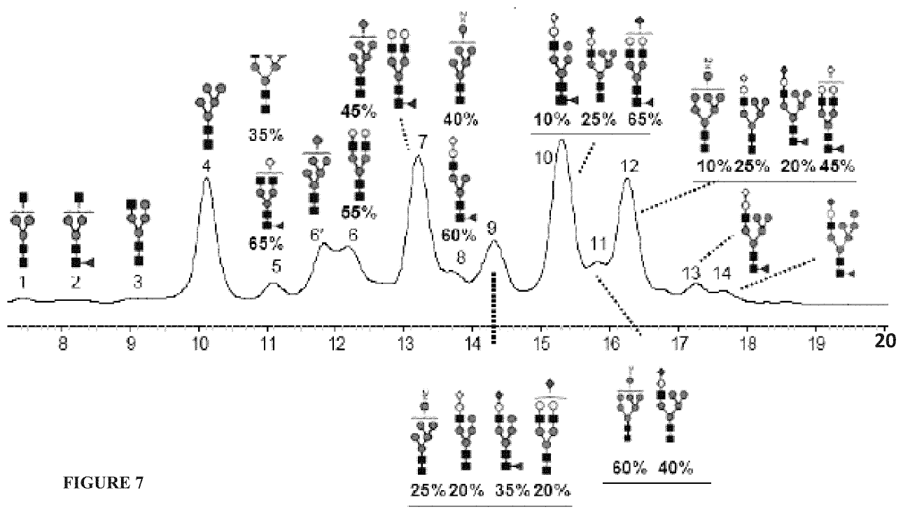

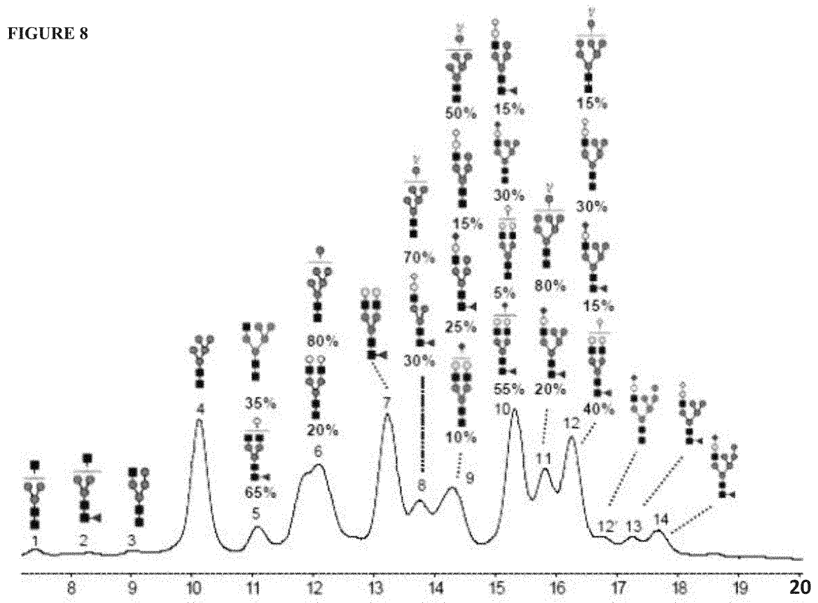

FIGS. 6-8 show the chromatograms of oligosaccharides (N-glycans) released from the transgenically produced adalimumab antibody in the milk of goat #2 as collected after 3 days of lactation (FIG. 6), 11 days of lactation (FIG. 7), and 21 days of lactation (FIG. 8).

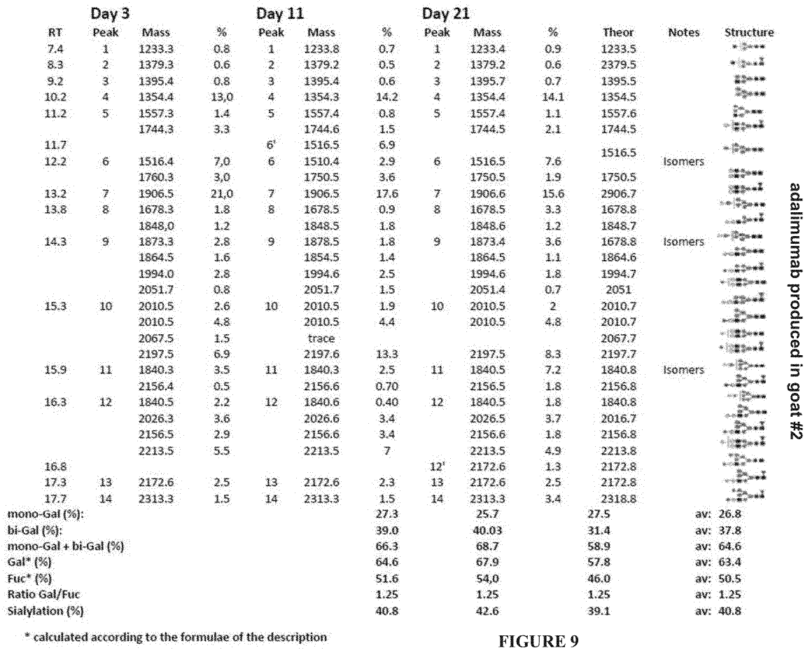

The relative percentages of all of the oligosaccharides (N-glycans) isolated from the adalimumab antibody produced in the milk of goat #2 are illustrated in FIG. 9.

FIG. 9 also shows a table of the overall percentage of mono-galactosylation, the percentage of bi-galactosylation, the total percentage of galactosylation (mono-galactosylation+bi-galactosylation), the percentage of galactosylation was calculated according to the formula indicated above, the percentage of fucosylation was calculated according to the formula indicated above, from the ratio of galactosylation to fucosylation and from the percentage of glycan structures having at least one sialic acid (% of sialylation). The results are also summarized in Table 2 below:

TABLE 2
oligosaccharides (N-glycans) isolated from the adalimumab antibodies
of goat #2

| | day 3 | day 11 | day 21 | average |
|---|---|---|---|---|
| mono-Gal (%): | 27.3 | 25.7 | 27.5 | 26.8 |
| bi-Gal (%): | 39.0 | 43.0 | 31.4 | 37.8 |
| mono-Gal + bi-Gal (%) | 66.3 | 68.7 | 58.9 | 64.6 |
| Gal* (%) | 64.6 | 67.9 | 57.8 | 63.4 |
| Fuc* (%) | 51.6 | 54.0 | 46.0 | 50.5 |
| Gal/Fuc | 1.25 | 1.25 | 1.25 | 1.25 |
| Sialylation (%) | 40.8 | 42.6 | 39.1 | 40.8 |

*calculated according to the formulae given in the description

### Example 2: Studies of the Binding of Transgenically Produced Adalimumab

FIG. 10 shows that transgenically produced adalimumab can bind soluble TNF-alpha coated in96-well plates. The transgenically produced adalimumab which is non-glycosylated is also capable of binding soluble TNF-alpha (data not shown).

FIG. 11 shows that the transgenically produced adalimumab binds the CD16 expressed by the natural killer (NK) cells. The binding was demonstrated in a competition experiment with the anti-CD16 antibody 3G8. The binding of the transgenically produced adalimumab to CD16 is stronger than the binding of an weakly galactosylated antibody to CD16 (data not shown).

### Example 3: Phagocytosis Test

### Material of Example 3

The following reagents were used:
transgenic anti-TNF Humira,
   non deglycosylated (i.e. glycosylated)
   deglycosylated
anti-TNF Humira, (Adalimumab, Abbott)
Polyvalent immunoglobulins IVIg (Tegeline, LFB)

Monocytes isolated from peripheral blood were thawed and differentiated into macrophages for 3 days in RPMI 1640+10% FCS+M-CSF at 50 ng/ml.

TNF-α was labelled with the Innova Biosciences Lightning-Link Rapid Conjugation System kit (green fluorescence) according to the manufacturer's instructions then incubated for 20 minutes at 4° C. with 10 μg/ml of anti-TNF alpha antibodies.

Phagocytosis was carried out for 3 hours at 4° C. and 37° C. by incubating the labelled TNF-α with the macrophages (1.10⁵ cells/well) in the presence or in the absence of 1 mg/ml of immunoglobulins IVIg.

The phagocytosis index analyzed by flow cytometry is estimated according to the following formula: MFI 37° C.-MFI 4° C. (Arbitrary unit).

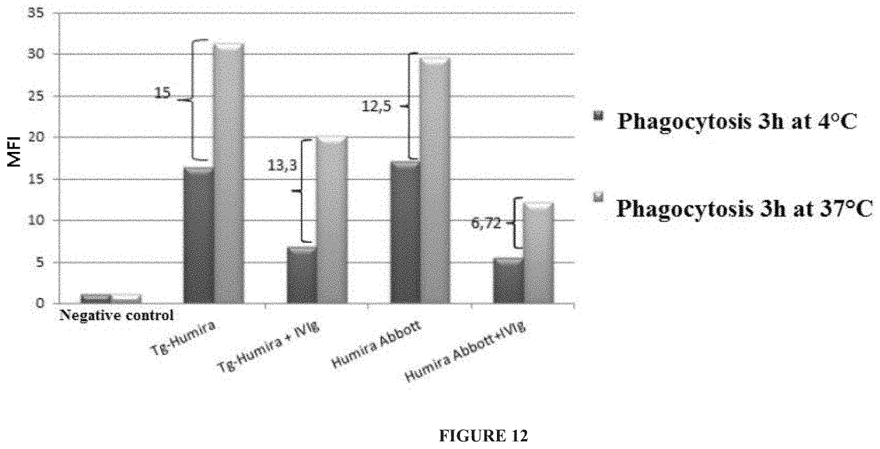

The results are shown in FIG. 12.

The macrophages alone (negative control) show an absence of fluorescence at 4° C. and 37° C.

In the presence of the non-deglycosylated transgenic adalimumab antibody (TG-Humira), TNF-α and macrophages, the phagocytosis value is 15 (MFI).

Under the same conditions, the addition of IVIg (1 mg/ml) induces inhibition of the binding of the TG-Humira antibody to the macrophages (FcR). This is observed at 4° C. and at 37° C. The phagocytosis is estimated at 13.3 (MFI)

In the presence of the Humira antibody from Abbott (commercial Humira), of TNF-α and of macrophages, the phagocytosis value is 12.5 (MFI).

Under the same conditions the addition of IVIg (1 mg/ml) induces inhibition of the binding of the Humira antibody from Abbott to the macrophages (FcR). This is observed at 4° C. and at 37° C. The phagocytosis is estimated at 6.72 (MFI)

These results thus show that the TG-Humira antibody induces phagocytosis of the TNF-α in the presence of CD16+ macrophages greater than that induced by the commercial adalimumab antibody (Humira, Abbott).

1. A method for the prevention or treatment of the early phases of inflammation comprising administering to a patient in need thereof a composition comprising monoclonal antibodies directed against a circulating proinflammatory cytokine, said antibodies have a high affinity for the FcγRIIIa receptor (CD16).

2. The method according to claim 1, wherein said antibodies of said composition have an affinity of at least equal to 2×10⁶ M⁻¹, at least equal to 2×10⁷ M⁻¹, 2×10⁸ M⁻¹ or 2×10⁹ M⁻¹, as determined
by Scatchard analysis or
BIAcore technology (Label-free surface plasmon resonance based technology) or
competition assay with an anti-CD16 antibody 3G8.

3. The method according to claim 1, wherein the fucose level of all of the antibodies of said composition is less than 60%, and preferably less than 50%.

4. (canceled)

5. The method according to claim 1, wherein the fucose level of all of the antibodies of said composition is less than 60%, and preferably less than 50%, and wherein each of the antibodies of said composition has, on the glycosylation site in position 297 of its heavy chains, one of the biantennary glycan forms selected from the following structures:

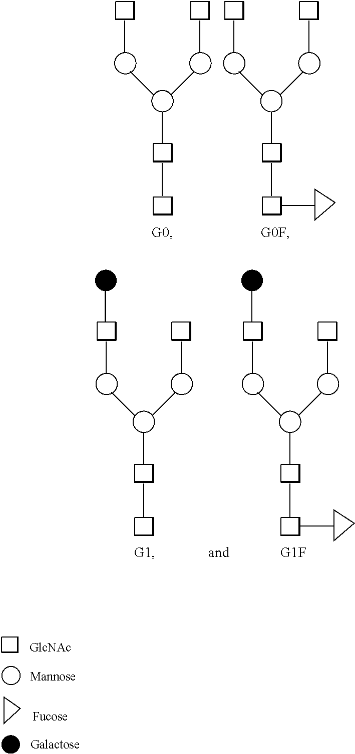

6. The method according to claim 5, wherein the G0F+G1F forms of the antibodies of said composition represent less than 50% of the glycan structures borne by the glycosylation site in position 297 of the heavy chain (Asn 297).

7. (canceled)

8. The method according to claim 1, wherein the fucose level of all of the antibodies of said composition is less than 60%, and preferably less than 50%,
and wherein each monoclonal antibody of said composition has an affinity for the FcγRIII receptors at least 1.5 times greater than that of a natural antibody directed against said circulating proinflammatory cytokine.

9. The method according to claim 1, wherein the fucose level of all of the antibodies of said composition is less than 60%, and preferably less than 50%,
and wherein said proinflammatory cytokine is selected from the group consisting of:
TNF-α,
IL-1β,
IL-6,
IL-8,
IL-10,
IL-12,
IL-17
IL-18, and
GM-CSF.

10. The method according to claim 1, wherein the fucose level of all of the antibodies of said composition is less than 60%, and preferably less than 50%,
and wherein said antibody of said composition has no properties of neutralization of said circulating proinflammatory cytokine.

11. The method according to claim 1, wherein the fucose level of all of the antibodies of said composition is less than 60%, and preferably less than 50%,
and wherein said antibody of said composition is used in doses varying from 0.05 mg/m² to 2000 mg/m².

12. The method according to claim 1, wherein the fucose level of all of the antibodies of said composition is less than 60%, and preferably less than 50%,
and wherein said antibody of said composition is in injectable form, or in spray form.

13. The method according to claim 1, wherein the fucose level of all of the antibodies of said composition is less than 60%, and preferably less than 50%,
and wherein said antibody of said composition is combined with a pharmaceutically acceptable vehicle.

14. The method according to claim 1, wherein the fucose level of all of the antibodies of said composition is less than 60%, and preferably less than 50%,
and wherein said composition is in combination with at least one anti-inflammatory agent.

15. The method according to claim 1, wherein the galactosylation level of all of the antibodies of said composition is at least 60%.

16. The method according to claim 1, wherein the galactosylation level of all of the antibodies of said composition is at least 60%,
and wherein the galactosylation level of all of the antibodies of said composition is at least 70%.

17. The method according to claim 1, wherein the galactosylation level of all of the antibodies of said composition is at least 60%,
and wherein the galactosylation level of all of the antibodies of said composition is at least 80%.

18. The method according to claim 1, wherein the galactosylation level of all of the antibodies of said composition is at least 60%,
and wherein the fucosylation level of all of the antibodies of said composition is at least 50%.

19. The method according to claim 1, wherein the galactosylation level of all of the antibodies of said composition is at least 60%,
and wherein the fucosylation level of all of the antibodies of said composition is at least 60%.

20. The method according to claim 1, wherein the galactosylation level of all of the antibodies of said composition is at least 60%,
and wherein the antibodies of said composition comprise mono-galactosylated N-glycans.

21. The method according to claim 1, wherein the galactosylation level of all of the antibodies of said composition is at least 60%,
and wherein the antibodies of said composition which comprise bi-galactosylated N-glycans.

22. The method according to claim 1, wherein the galactosylation level of all of the antibodies of said composition is at least 60%,
and wherein the ratio of the galactosylation level of the antibodies of said composition to the fucosylation level of the antibodies of said composition is from 1.0 to 1.4.

23. The method according to claim 1, wherein the galactosylation level of all of the antibodies of said composition is at least 60%,
and wherein at least 35% of the antibodies of said composition comprise bi-galactosylated N-glycans and at least 25% of the antibodies comprise mono-galactosylated N-glycans.

24. The method according to claim 1, wherein the antibody of said composition is produced in the mammary epithelial cells of a non-human mammal.

25. The method according to claim 1, wherein the antibody of said composition is produced in a transgenic non-human mammal, in particular in a goat, a sheep, a bison, a camel, a cow, a pig, a rabbit, a buffalo, a horse, a rat, a mouse or a llama.

26. The method according to claim 1, wherein said composition also comprises milk.

27. The method according to claim 1, wherein said composition also comprises a pharmaceutically acceptable vehicle.

28. The method according to claim 1, wherein the antibodies of said composition contain an oligomannose.

29. The method according to claim 1, wherein the antibodies of said composition contain an oligomannose,
and wherein at least 30% of the antibodies of said composition contain at least one oligomannose.

30. The method according to claim 1, wherein the antibodies of said composition contain an oligomannose,
and wherein the N-glycans of the antibodies of said composition have a highly mannosylated glycosylation profile.

31. The method according to claim 1, wherein the antibodies of said composition contain an oligomannose, and wherein
the N-glycans of the antibodies of said composition have a highly mannosylated glycosylation profile, and
at least one chain of the antibodies of said composition contains an oligomannose and is not fucosylated.

32. The method according to claim 1, wherein the antibodies of said composition contain an oligomannose,
and wherein a major N-glycan of the antibodies is not fucosylated.

33. The method according to claim 1, wherein the antibodies of said composition contain an oligomannose,
and wherein
a major N-glycan of the antibodies of said composition is not fucosylated, and
said major N-glycan of the antibodies of said composition is a non-fucosylated oligomannose.

34. The method according to claim 1, wherein the antibodies of said composition contain an oligomannose,
and wherein
a major N-glycan of the antibodies of said composition is not fucosylated, and
said major N-glycan of the antibodies of said composition is a non-fucosylated Man5.

35. The method according to claim 1, wherein the antibodies of said composition contain an oligomannose,
and wherein less than 40% of the N-glycans of the antibodies of said composition contain fucose.

36. The method according to claim 1, wherein the antibodies of said composition contain an oligomannose,
and wherein the antibodies of said composition contain no fucose.

37. The method according to claim 1, wherein the antibodies of said composition contain an oligomannose, and wherein
at least 60% of the N-glycans of the antibodies of said composition contain a non-fucosylated oligomannose, and
less than 40% of the N-glycans of the antibodies of said composition contain fucose.

38. The method according to claim 1, wherein the fucose level of all of the antibodies of said composition is less than 60%, and preferably less than 50%,
and wherein each of the antibodies of said composition has, on the glycosylation site in position 297 of its heavy chains, one of the biantennary glycan forms selected from the following structures: 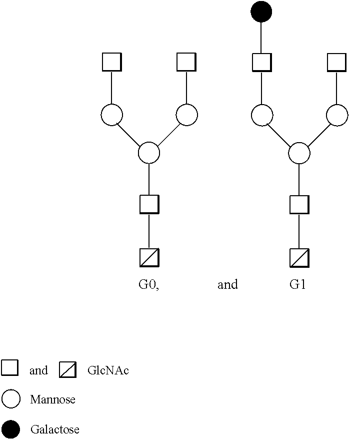
the GlcNAc represented by [symbol] in the above G0 and G1 structures being capable of being fucosylated.

39. The method according to claim 38, wherein the G0+G1+G0F+G1F forms of the antibodies of said composition represent more than 60% of said glycan structures and preferably more than 80% of the glycan structures borne by the glycosylation site in position 297 of the heavy chain (Asn 297).
